SEQUENCE LISTING (27 sequences)

SEQ ID NO: 1 - DNA, 102 bp, Homo sapiens
gtcttcctgctgatgcaatccgctaggtcgcgagtctccgccgcgagagggccggtctgcaatccagcccgccacgtgtactcgccgccgcctcgggcactg

SEQ ID NO: 2 - DNA, 95 bp, Homo sapiens
gacgacgcaccctctccgtgtcccgctctgcgcccttctgcgcgccccgctccctgtaccggagcagcgatccgggaggcggccgagaggtgcgc

SEQ ID NO: 3 - DNA, 29 bp, Artificial Sequence
  Primer
gtttttttgttgatgtaattcgttaggtc

SEQ ID NO: 4 - DNA, 24 bp, Artificial Sequence
  Primer
gacgacgtatttttttcgtgtttc

SEQ ID NO: 5 - DNA, 20 bp, Artificial Sequence
  Primer
caatacccgaaacgacgacg

SEQ ID NO: 6 - DNA, 16 bp, Artificial Sequence
  Primer
gcgcacctctcgaccg

SEQ ID NO: 7 - DNA, 21 bp, Artificial Sequence
  Probe
  /HEX/ (1)..(1): 5′ HEX (hexachlorofluorescein)
  /BHQ/ (21)..(21): 3′ BHQ (Black Hole quencher)
ttcgtcgcgagagggtcggtt

SEQ ID NO: 8 - DNA, 27 bp, Artificial Sequence
  Probe
  /FAM/ (1)..(1): 5′ 6-FAM (fluorescein)
  /ZEN/ (9)..(10): ZEN Internal Quencher
  /3IABkFQ/ (27)..(27): 3′ IABkFQ (Iowa Black FQ quencher)
tttgtatyggagtagygattygggagg

SEQ ID NO: 9 - DNA, 102 bp, Homo sapiens
cagtgcccgaggcggcggcgagtacacgtggcgggctggattgcagaccggccctctcgcggcggagactcgcgacctagcggattgcatcagcaggaagac

SEQ ID NO: 10 - DNA, 102 bp, Artificial Sequence
  Bisulphite converted sequence
tagtgttcgaggcggcggcgagtatacgtggcgggttggattgtagatcggttttttcgcggcggagattcgcgatttagcggattgtattagtaggaagat

SEQ ID NO: 11 - DNA, 102 bp, Homo sapiens; identical to SEQ ID NO: 1

SEQ ID NO: 12 - DNA, 102 bp, Artificial Sequence
  Bisulphite converted sequence
gtttttttgttgatgtaattcgttaggtcgcgagttttcgtcgcgagagggtcggtttgtaatttagttcgttacgtgtattcgtcgtcgtttcgggtattg

SEQ ID NO: 13 - DNA, 21 bp, Artificial Sequence
  Probe
ttcgtcgcgagagggtcggtt

SEQ ID NO: 14 - DNA, 26 bp, Artificial Sequence
  Probe
tttttgttgatgtaattcgttaggtc

SEQ ID NO: 15 - DNA, 20 bp, Artificial Sequence
  Probe
attacaaaccgaccctctcg

SEQ ID NO: 16 - DNA, 87 bp, Homo sapiens
agatcccaagggtcgtagcccctggccgtgtggaccgggtctgcggctgcagagcgcggtcccggctgcagcaagacctggggcagt

SEQ ID NO: 17 - DNA, 19 bp, Artificial Sequence
  Probe
agatcccaagggtcgtagc

SEQ ID NO: 18 - DNA, 18 bp, Artificial Sequence
  Probe
actgccccaggtcttgct

SEQ ID NO: 19 - DNA, 87 bp, Homo sapiens
actgccccaggtcttgctgcagccgggaccgcgctctgcagccgcagacccggtccacacggccaggggctacgacccttgggatct

SEQ ID NO: 20 - DNA, 95 bp, Artificial Sequence
  Bisulphite converted sequence
gacgacgtatttttttcgtgtttcgttttgcgtttttttgcgcgtttcgttttttgtatcggagtagcgattcgggaggcggtcgagaggtgcgt

SEQ ID NO: 21 - DNA, 26 bp, Artificial Sequence
  Probe
tttgtatcggagtagcgattcgggag

SEQ ID NO: 22 - DNA, 95 bp, Homo sapiens
gcgcacctctcggccgcctcccggatcgctgctccggtacagggagcggggcgcgcagaagggcgcagagcgggacacggagagggtgcgtcgtc

SEQ ID NO: 23 - DNA, 95 bp, Artificial Sequence
  Bisulphite converted sequence
gcgtatttttcggtcgtttttcggatcgttgtttcggtatagggagcggggcgcgtagaagggcgtagagcgggatacggagagggtgcgtcgtt

SEQ ID NO: 24 - DNA, 92 bp, Artificial Sequence
  Bisulphite converted sequence
ccggagttgcggctgagacgcgcgccgcgcgagccgggggactcggcgacggggcggggacgggacgacgcaccctctccgtgtcccgctct

SEQ ID NO: 25 - DNA, 17 bp, Artificial Sequence
  Probe
ggagttgcggctgagac

SEQ ID NO: 26 - DNA, 17 bp, Artificial Sequence
  Probe
agagcgggacacggaga

SEQ ID NO: 27 - DNA, 92 bp, Homo sapiens
agagcgggacacggagagggtgcgtcgtcccgtccccgccccgtcgccgagtcccccggctcgcgcggcgcgcgtctcagccgcaactccgg

(19) United States
(12) Patent Application Publication
(10) Pub. No.: US 2025/0043354 A1
(43) Pub. Date: Feb. 6, 2025

(54) SCREENING METHOD

(71) Applicants: Flinders University of South Australia, South Australia (AU); Clinical Genomics Pty. Ltd., North Ryde, New South Wales (AU)

(72) Inventors: Graeme Paul YOUNG, South Australia (AU); Erin SYMONDS, South Australia (AU); Lawrence C LAPOINTE, North Ryde, New South Wales (AU); Susanne Kartin PEDERSEN, North Ryde, New South Wales (AU); Damian James HUSSEY, North Ryde, New South Wales (AU)

(21) Appl. No.: 17/310,116

(22) PCT Filed: Jan. 15, 2020
(86) PCT No.: PCT/US2020/013623

§ 371 Date: Jul. 16, 2021

(30) Foreign Application Priority Data
Jan. 18, 2019 (AU) 2019200359

(51) Int. Cl. C12Q 1/6886

(52) U.S. Cl. CPC C12Q 1/6886; C12Q 2600/118; C12Q 2600/154

(57) ABSTRACT

The present disclosure is related to methods of screening for the onset or predisposition to the onset of or monitoring an esophageal or gastric neoplasm in an individual, said method comprising assessing the methylation status of a selected DNA region in a biological sample from said individual wherein a higher level of methylation of at least one of the DNA regions relative to control levels is indicative of an esophageal or gastric neoplasm or a predisposition to the onset of a esophageal or gastric neoplasm.

### CROSS-REFERENCE TO RELATED APPLICATIONS

This application is a U.S. National Phase Application of PCT International Application Number PCT/US2020/013623, filed on Jan. 15, 2020, designating the United States of America and published in the English language, which is an International Application of and claims the benefit of priority to Australian Application No. 2019200359, filed on Jan. 18, 2019. The disclosures of the above-referenced applications are hereby expressly incorporated by reference in their entireties.

### FIELD

Aspects of the present invention generally relate to methods of screening for the onset, predisposition to the onset and/or progression of an esophageal or gastric neoplasm. More particularly, some embodiments relate to methods of screening for the onset, predisposition to the onset and/or progression of an esophageal or gastric neoplasm by screening for changes to the methylation level or patterns of methylation of a panel of selected genes. The embodiments described herein are useful in a range of applications including, but not limited to, those relating to the diagnosis and/or monitoring of esophageal or gastric neoplasms, such as esophageal or gastric adenocarcinomas.

### BACKGROUND

Gastrointestinal cancer refers to malignant conditions of the gastrointestinal tract (GI tract) and accessory organs of digestion, including the esophagus, stomach, biliary system, pancreas, small intestine, large intestine, rectum and anus. The symptoms relate to the organ affected and can include obstruction (leading to difficulty swallowing or defecating), abnormal bleeding or other associated problems. Overall, the GI tract and the accessory organs of digestion (pancreas, liver, gall bladder) are responsible for more cancers and more deaths from cancer than any other system in the body.

Despite the fact that the organs of the GI tract share a common functionality, in that they all contribute in some way to the digestive processes, they are nevertheless each entirely distinct and unique organs, both in terms of their tissue structure and function. Accordingly, there is significant variation in the rates, causes, etiology and prognosis of different gastrointestinal cancers and they share no more commonality in terms of etiology than they share with non-gastrointestinal cancers.

Invasive cancers that are confined within the wall of the organs which form part of the GI tract (e.g. TNM stages I and II) are usually curable with surgery. If untreated, they spread to regional lymph nodes (e.g. TNM stage III), where they may be curable by a combination of surgery and chemotherapy. Cancer that metastasizes to distant sites (e.g. TNM stage IV) is usually not curable, although chemotherapy can extend survival, and in rare cases, surgery and chemotherapy together have seen patients through to a cure.

Esophageal and gastric cancers are both common and generally present with poor prognosis, primarily because patients present most often after significant disease progression has occurred and survival is therefore poor. Esophageal cancer is the sixth-most-common cancer in the world, and its incidence is increasing, with three to five males being affected for each female. Cancer of the esophagus is often detected late in as much as there are typically no early symptoms. Nevertheless, if the cancer is caught soon enough, patients can have a five-year survival rate of 90% or above. By the time esophageal cancer is usually detected, though, it has spread beyond the esophageal wall, and the survival rate drops significantly. In China, the overall five-year survival rate for advanced esophageal cancer is about 20%, and in the United States it is about 15%. Gastric cancer, is the fourth-most-common type of cancer and the second-highest cause of cancer death globally. The most common type of gastric cancer is adenocarcinoma. As with esophageal cancer, diagnosis is often quite late, although not necessarily due to the absence of early symptoms but, rather, due to the fact that many of the symptoms may occur in other diseases as well, and hence symptoms may not be conclusively diagnostic of gastric cancer until the disease is advanced.

Esophageal cancer is cancer arising from the esophagus—the lengthy muscular tube that runs between the throat and the stomach. Later stage symptoms often include difficulty in swallowing and weight loss. Other symptoms may include pain when swallowing, a hoarse voice, enlarged lymph nodes around the collarbone, a dry cough, and possibly coughing up or vomiting blood. The two main sub-types of the disease are esophageal squamous-cell carcinoma (ESCC), which is more common in the developing world, and esophageal adenocarcinoma (EAC), which is more common in the developed world. A number of less common types also occur. Squamous-cell carcinoma arises from the epithelial cells that line the esophagus. Adenocarcinoma tends to arise from glandular cells present in the lower third of the esophagus, usually when squamous cells have transformed to glandular cells (a condition known as Barrett's esophagus). Although the causes of this transformation are only partially understood, the long-term erosive effects of gastroesophageal reflux disease (GERD) or bile reflux have been strongly linked to this type of cancer. Longstanding GERD can induce a change of cell type in the lower portion of the esophagus in response to erosion of its squamous lining. This phenomenon, seems to appear about 20 years later in women than in men, possibly due to hormonal factors.

Gastric cancer generally develops in the lining of the stomach. Early symptoms may include indigestion, upper abdominal pain, nausea and loss of appetite. Later signs and symptoms may include weight loss, yellowing of the skin and whites of the eyes, vomiting, difficulty swallowing and blood in the stool among others. These are all quite general symptoms which are attributable to a multitude of causes.

A particularly common cause of gastric cancer is infection by the bacterium Helicobacter pylori, which accounts for more than 60% of cases. About 10% of cases run in families, and between 1% and 3% of cases are due to genetic syndromes inherited from a person's parents such as hereditary diffuse gastric cancer. Gastric cancer tends to develop in stages over years.

Prognosis and treatment planning for esophageal cancer are dependent on the ability to make a diagnosis and establish a clinical stage. A barium swallow study is often performed. It provides cancer suspicion and tumor localization for later endoscopy. Esophagoscopy, however, is necessary for making a diagnosis. It allows for direct visualization and evaluation of Barrett's esophagus and tumors, and can provide a tissue diagnosis with a biopsy. Single biopsies of suspicious lesions in this setting are 93% accurate. The accuracy can improve to 100% with the addition of multiple biopsies (seven) and brushings. An ultrasound probe combined with conventional endoscopy (endoscopic ultrasound) provides the most accurate estimate of disease stage by the tumor-node-metastasis criteria established by the American Joint Committee on Cancer. Ultrasound does so by evaluating the five layers of the esophageal wall with 85% accuracy and by detecting abnormal mediastinal lymph node metastasis with approximately 80% accuracy. EUS guided fine needle aspiration enhances this accuracy.

A clear method for diagnosing and screening gastric cancer is important since its presentation often mimics other disease processes and there is only a small window of time where the disease is potentially curable. Each different diagnostic modality exhibits strengths and weakness. Although invasive and costly, endoscopy is currently the most specific and sensitive method for obtaining a definitive diagnosis besides laparotomy, and has replaced barium contrast radiographs due to its ability to biopsy. It allows for direct visualization of lesions and the opportunity to obtain tissue samples. Endoscopy with seven biopsies of the margin and base of gastric ulcers has a sensitivity of 98% for diagnosing gastric cancers of all stages, compared to barium studies with a sensitivity of only 14% in early gastric cancers. Barium continues to have a major role in diagnosing of linitis plastica, a diffuse type gastric cancer which is difficult to appreciate with endoscopy. Brush biopsies are performed in areas at risk of bleeding.

Due to demographic variability and recent changes in disease incidence, much emphasis has been placed on studying risk factors for both esophageal and gastric cancers. However, despite increasing understanding of these diseases, low survival rates persist. Esophageal and gastric cancers can take many years to develop and early detection of these cancers greatly improves the prognosis. However, the combination of an absence of symptoms, or very non-specific symptoms, together with there being predominantly only invasive and expensive diagnostic techniques which are reliable, tends to result in actual diagnosis occurring at a very late stage. Even modest efforts to implement earlier stage screening methods would result in a drop in cancer deaths. However, the absence of a simple, non-invasive and cost-effective screening method prevents this. Most of the more sensitive tests are quite invasive and expensive and therefore uptake by patients is low. There is therefore an ongoing need to develop simpler and more informative diagnostic protocols or aids to diagnosis that enable one to direct the more invasive or expensive diagnostic methods to people more likely to have developed carcinomas. A simple and accurate screening test would enable much more widely applicable screening systems to be set up.

In work leading up to the present invention it has been determined that changes to the methylation of the BCAT1 and/or IKZF1 genes is indicative of the development of esophageal and gastric neoplasms, such as carcinomas. Still further, the identification of specific genomic DNA cytosine nucleotides, which become hypermethylated, has enabled the development of very simple and specific amplification reactions for routine use in the context of diagnosis. Diagnosis can therefore be made based on screening for one or both of these two differentially methylated genes. These data are surprising when one considers that although BCAT1 and IKZF1 are known to be hypermethylated in colorectal neoplasia, these are not pan cancer markers and have been definitively shown to remain unchanged in neoplastic tissues which exhibit some of the same histological characteristics as esophageal and gastric tissue. For example, each of gastric, breast and prostate tissue are glandular in nature. The glandular cells of each of these three tissues have been shown to give rise to adenocarcinomas, yet breast and prostate adenocarcinomas have repeatedly been demonstrated to show no change to the methylation of BCAT1 and IKZF1. As between esophageal and gastric tissue, these organs are entirely distinct and therefore any similarity in neoplastic marker expression would not be expected and is entirely serendipitous. The extent of their dissimilarity is evidenced not only by the differences in their histology but, still further, functional differences such as the fact that Helicobacter pylori infection is not a risk factor for esophageal adenocarcinoma, and actually appears to be protective, despite being a common cause of GERD and a risk factor for gastric cancer. In fact, this infection seems to be associated with a reduced risk of esophageal adenocarcinoma of as much as 50%. Accordingly, the inventors have identified two genes which facilitate the diagnosis of neoplasia and/or the monitoring of conditions characterized by neoplasia in the esophagus and stomach.

### SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains nucleotide sequence information prepared using the programme Patent In Version 3.5, presented herein after the bibliography. Each nucleotide sequence is identified in the sequence listing by the numeric indicator <210> followed by the sequence identifier (e.g. <210>1, <210>2, etc). The length, type of sequence (DNA, etc) and source organism for each sequence is indicated by information provided in the numeric indicator fields <211>, <212> and <213>, respectively. Nucleotide sequences referred to in the specification are identified by the indicator SEQ ID NO: followed by the sequence identifier (e.g. SEQ ID NO:1, SEQ ID NO:2, etc.). The sequence identifier referred to in the specification correlates to the information provided in numeric indicator field <400> in the sequence listing, which is followed by the sequence identifier (e.g. <400>1, <400>2, etc). That is, SEQ ID NO:1 as detailed in the specification correlates to the sequence indicated as <400>1 in the sequence listing.

One aspect of the present invention is directed to a method of screening for the onset or predisposition to the onset of or monitoring an esophageal or gastric neoplasm in an individual, said method comprising assessing the methylation status of a DNA region selected from:
(i) the region, including 2 kb upstream of the transcription start site, defined by Hg19 coordinates:
   (1) chr12:24962958 . . . 25102393; and/or
   (2) chr7:50344378 . . . 50472798; or
(ii) the gene region, including 2 kb upstream, of:
   (1) BCAT1; and/or (2) IKZF1
      in a biological sample from said individual wherein a higher level of methylation of at least one of the DNA regions of group (i) and/or (ii) relative to control levels is indicative of an esophageal or gastric neoplasm or a predisposition to the onset of an esophageal or gastric neoplastic state.

In one embodiment, said method is directed to screening either BCAT1 or IKZF1 in a biological sample.

In another embodiment, said method is directed screening both BCAT1 and IKZF1 in a biological sample.

In still another embodiment, said method is directed to screening both BCAT1 and IKZF1 but wherein only one of said genes exhibits a higher level of methylation.

In a further embodiment, said method is directed to screening both BCAT1 and IKZF1 but wherein both of said genes exhibit a higher level of methylation.

In still another embodiment said control level is a non-neoplastic level.

In yet another embodiment said control level is the level of a previously screened biological sample from said individual.

In still another embodiment, the neoplasm is malignant, such as a carcinoma.

In a further embodiment, the neoplasm is non-malignant, such as an adenoma.

In yet another further embodiment said neoplasm is an esophageal or gastric adenoma or adenocarcinoma.

The subregions which have been determined to exhibit particular utility are:
(1) BCAT1 subregions chr12:25101992-25102093 (SEQ ID NO:1 or corresponding minus strand) and chr12:25101909-25101995 (SEQ ID NO:2 or corresponding minus strand); and
(2) IKZF1 subregions: chr7:50343867-50343961 (SEQ ID NO:3 or corresponding minus strand) and chr7:50343804-5033895 (SEQ ID NO:4 or corresponding minus strand).

Without limiting the present invention to any one theory or mode of action, the skilled person may screen one or more subregions for each gene marker.

In one embodiment, the methylation marker subregions, which are tested are:
(1) The BCAT1 subregion defined by SEQ ID NO:1 or SEQ ID NO:2 or corresponding minus strand; and
(2) The IKZF1 subregion defined by SEQ ID NO:3 or SEQ ID NO:4 or corresponding minus strand;

To the extent that the method of the present invention includes analyzing IKZF1, one may assess the methylation of one or more of:

| | | |
|---|---|---|
| chr7: 50343869 | chr7: 50343872 | chr7: 50343883 |
| chr7: 50343889 | chr7: 50343890 | chr7: 50343897 |
| chr7: 50343907 | chr7: 50343909 | chr7: 50343914 |
| chr7: 50343934 | chr7: 50343939 | chr7: 50343950 |
| chr7: 50343959 | chr7: 50343805 | chr7: 50343822 |
| chr7: 50343824 | chr7: 50343826 | chr7: 50343829 |
| chr7: 50343831 | chr7: 50343833 | chr7: 50343838 |
| chr7: 50343847 | chr7: 50343850 | chr7: 50343858 |
| chr7: 50343864 | chr7: 50343869 | chr7: 50343872 |
| chr7: 50343890 | | |

or a corresponding cytosine at position n+1 on the opposite DNA strand.

In another embodiment said sample is a surgical resection, tissue biopsy, saliva, urine or blood sample (e.g. whole blood, serum, plasma, exosomes, or buffy coat).

Still more preferably, said method is directed to screening the methylation of plasma derived cell-free DNA, such as circulating tumor DNA.

Another aspect of the present invention is directed to a method of screening for the onset or predisposition to the onset of or monitoring an esophagus or gastric neoplasm in an individual, said method comprising assessing the level of expression of a DNA region selected from:
(i) the region, including 2 kb upstream of the transcription start site, defined by Hg19 coordinates:
   (1) chr12:24962958 . . . 25102393; and/or
   (2) chr7:50344378 . . . 50472798; or
(ii) the gene region, including 2 kb upstream of:
   (1) BCAT1; and/or (2) IKZF1
      in a biological sample from said individual wherein a lower level of expression of at least one of the DNA regions of group (i) and/or (ii) relative to control levels is indicative of an esophagus or gastric neoplasm or a predisposition to the onset of a neoplastic state.

A related aspect of the present invention provides a molecular array, which array comprises a plurality of:
(i) nucleic acid molecules comprising a nucleotide sequence corresponding to any two or more of the neoplastic marker DNA hereinbefore described or a sequence exhibiting at least 80% identity thereto or a functional derivative, fragment, variant or homologue of said nucleic acid molecule; or
(ii) nucleic acid molecules comprising a nucleotide sequence capable of hybridizing to any one or more of the sequences of (i) under medium stringency conditions or a functional derivative, fragment, variant or homologue of said nucleic acid molecules; or
(iii) nucleic acid probes or oligonucleotides comprising a nucleotide sequence capable of hybridizing to any two or more of the sequences of (i) under medium stringency conditions or a functional derivative, fragment, variant or homologue of said nucleic acid molecule; or
(iv) probes capable of binding to any two or more of the proteins encoded by the nucleic acid molecules of (i) or a derivative, fragment or, homologue thereof
wherein the level of expression of said marker genes of (i)-(iii) or proteins of (iv) is indicative of the neoplastic state of a cell or cellular subpopulation derived from the esophagus or stomach.

Accordingly, some embodiments provided herein are described by way of the following numbered alternatives:

1. A method of screening for the onset or predisposition to the onset of or monitoring an esophageal or gastric neoplasm in an individual, said method comprising assessing the methylation status of a DNA region selected from:
(i) the region, including 2 kb upstream of the transcription start site, defined by Hg19 coordinates:
   (1) chr12:24962958 . . . 25102393; and/or
   (2) chr7:50344378 . . . 50472798
   or
(ii) the gene region, including 2 kb upstream of any two or more of:
   (1) BCAT1 and/or (2) IKZF1
      in a biological sample from said individual wherein a higher level of methylation of at least one of the DNA regions of group (i) and/or (ii) relative to control levels is indicative of an esophageal or gastric neoplasm or a predisposition to the onset of a esophageal or gastric neoplasm and optionally providing, guiding, or advising said individual to receive a colonoscopy, surgical removal of malignant tissue, and/or radiation, chemotherapy, or immunotherapy when the level of methylation is higher than the control levels.

2. A method of screening for the onset or predisposition to the onset of or monitoring an esophageal or gastric neoplasm in an individual, said method comprising assessing the level of expression of a DNA region selected from:
(i) the region, including 2 kb upstream of the transcription start site, defined by Hg19 coordinates:
   (1) chr12:24962958 . . . 25102393; and/or
   (2) chr7:50344378 . . . 50472798;
   or
(ii) the gene region, including 2 kb upstream of:
   (1) BCAT1 and/or (2) IKZF1
      in a biological sample from said individual wherein a lower level of expression of at least one of the DNA regions of group (i) and/or (ii) relative to control levels is indicative of an esophageal or gastric neoplasm or a predisposition to the onset of a neoplasm and, optionally providing, guiding, or advising said individual to receive a colonoscopy, surgical removal of malignant tissue, and/or radiation, chemotherapy, or immunotherapy when the level of methylation is lower than the control levels.

3. The method according to alternatives 1 or 2 wherein said method is directed to screening either BCAT1 or IKZF1 in said biological sample.

4. The method according to alternatives 1 or 2, said method is directed to screening both BCAT1 and IKZF1 in said biological sample.

5. The method according to alternative 4 wherein only one of said BCAT1 and IKZF1 exhibits modulated methylation or expression.

6. The method according to alternative 4 wherein both of said BCAT1 and IKZF1 exhibit modulated methylation or expression.

7. The method according to any one of alternatives 1-6 wherein the neoplasm is malignant.

8. The method according to alternative 7 wherein said malignant neoplasm is an adenocarcinoma.

9. The method according to any one of alternatives 1-6 wherein said neoplasm is not malignant.

10. The method according to alternative 9 wherein said non-malignant neoplasm is an adenoma.

11. The method according to any one of alternatives 1 to 10 wherein said control level is a non-neoplastic level.

12. The method according to any one of alternatives 1 to 10 wherein said control level is the level of a previously screened biological sample from said individual.

13. The method according to alternative 12 wherein a decrease in the level of methylation relative to said control level or an increase in the level of DNA expression relative to said control level is indicative of the clearing of the neoplasm.

14. The method according to any one of alternatives 1 to 13 wherein said neoplasm is a gastric neoplasm.

15. The method according to any one of alternatives 1 to 13 wherein said neoplasm is an esophageal neoplasm.

16. The method according to any one of alternatives 1 to 15 wherein said biological sample is a surgical resection, tissue biopsy, saliva, urine or blood sample.

17. The method according to alternative 16 wherein said blood sample is whole blood, serum, plasma, exosomes, or buffy coat.

18. The method according to alternative 17 wherein the DNA methylation screening is directed to cell free DNA.

19. The method according to alternative 18 wherein said cell free DNA is circulating tumor DNA.

20. The method according to alternative 1 wherein said methylation is assessed in one or more chromosomal subregions selected from:
(1) BCAT1 subregions chr12:25101992-25102093 (SEQ ID NO:11 or corresponding minus strand) and chr12:25101909-25101995 (SEQ ID NO:16 or corresponding minus strand)
(2) IKZF1 subregions: chr7:50343867-50343961 (SEQ ID NO:2 or corresponding minus strand) and chr7:50343804-5033895 (SEQ ID NO:24 or corresponding minus strand)

21. The method according to alternative 20, said method comprising assessing the methylation of one or more cytosine residues selected from:

(IKZF1)

| | | |
|---|---|---|
| chr7: 50343869 | chr7: 50343872 | chr7: 50343883 |
| chr7: 50343889 | chr7: 50343890 | chr7: 50343897 |
| chr7: 50343907 | chr7: 50343909 | chr7: 50343914 |
| chr7: 50343934 | chr7: 50343939 | chr7: 50343950 |
| chr7: 50343959 | chr7: 50343805 | chr7: 50343822 |
| chr7: 50343824 | chr7: 50343826 | chr7: 50343829 |
| chr7: 50343831 | chr7: 50343833 | chr7: 50343838 |
| chr7: 50343847 | chr7: 50343850 | chr7: 50343858 |
| chr7: 50343864 | chr7: 50343869 | chr7: 50343872 |
| chr7: 50343890 | | |

or a corresponding cytosine at position n+1 on the opposite DNA strand.

22. The method according to alternative 2 wherein said level of expression is mRNA expression or protein expression.

23. The method according to any one of alternatives 1 to 22 wherein said mammal is a human.

### BRIEF DESCRIPTION OF THE DRAWINGS

In order to describe the manner in which the above-recited and other advantages and features of the invention can be obtained, a more particular description will be rendered by reference to specific embodiments thereof which are illustrated in the appended drawings. Understanding that these drawings depict only typical embodiments and are not therefore to be considered to be limiting of its scope, the invention will be described and explained with additional specificity and detail through the use of the accompanying drawings.

FIG. 1 is a graphical representation of % methylation levels of BCAT1 and IKZF1 in neoplastic and non-neoplastic colorectal, esophageal, prostate, and breast tissue.

### DETAILED DESCRIPTION

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2, 1, or 0.5% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques, many of which are described below for the purpose of illustration. Such techniques are known and commonly used by a person of ordinary skill in the art.

The present disclosure refers to staging of a cancer or tumor to determine the progression of spread of the cancer or tumor within a patient. A commonly recognized standard for the classification of solid tumors is the TNM classification standard, which distinguishes a tumor based on the size of the tumor (T), extent of spread to lymph nodes (N), and metastasis (M). These classifications are further grouped into stages, wherein stage 0 growths are non-malignant neoplasms, stage I and II tumors are locally contained, stage III tumors have spread to nearby lymph nodes, and stage IV tumors have metastasized. While the TNM standard is a widely used method of classification, alternative or modified standards which may represent the behavior of a particular cancer type may also be employed. Accordingly, while these standards are useful for determining progression, prognosis of early or late stages of a cancer or tumor are independent from a specific classification.

The present invention is predicated, in part, on the elucidation of DNA methylation status, which characterizes esophageal and gastric neoplasms. This finding has now facilitated the development of routine approaches for screening for the onset or predisposition to the onset of or monitoring an esophageal and gastric neoplasm based on increased methylation of certain genes relative to control levels. In accordance with the present invention, it has been determined that the genes BCAT1 and/or IKZF1 may be altered, in terms of differential changes to methylation, depending on whether or not the cell in issue is neoplastic or not. It should be understood that the genes at issue are described herein both by reference to their name and their chromosomal coordinates. To the extent that chromosomal coordinates corresponding to gene names are listed, these are consistent with the human genome database version Hg19, which was released in February 2009 (herein referred to as “Hg19 coordinates”). It should also be understood that BCAT1 and IKZF1 are also referred to herein as a “panel” of markers.

Accordingly, one aspect of the present invention is directed to a method of screening for the onset or predisposition to the onset of or monitoring an esophageal or gastric neoplasm in an individual, said method comprising assessing the methylation status of a DNA region selected from:
(i) the region, including 2 kb upstream of the transcription start site, defined by Hg19 coordinates:
   (1) chr12:24962958 . . . 25102393; and/or
   (2) chr7:50344378 . . . 50472798; or
(ii) the gene region, including 2 kb upstream, of:
   (1) BCAT1; and/or (2) IKZF1
      in a biological sample from said individual wherein a higher level of methylation of at least one of the DNA regions of group (i) and/or (ii) relative to control levels is indicative of an esophageal or gastric neoplasm or a predisposition to the onset of an esophageal or gastric neoplastic state.

To the extent that the present invention is directed to embodiments of the method in which “one of” the DNA regions (markers) exhibits a higher level of methylation, these embodiments are designed to achieve results based on either one of the markers in the panel is hypermethylated. It should be understood that it need not be the same marker, which is hypermethylated in each sample. Rather, it is simply that one of the markers, which forms part of the panel, is hypermethylated. It should also be understood that in relation to some samples, both of the markers of the panel may be hypermethylated.

Reference to “esophagus”, commonly known as the food pipe or gullet (gut), should be understood as a reference to the organ through which food passes from the pharynx to the stomach. Without limiting the present invention to any one theory or mode of action, the esophagus is a fibromuscular tube, about 25 cm long in adults, which travels behind the trachea and heart, passes through the diaphragm and empties into the uppermost region of the stomach. The esophagus is one of the upper parts of the digestive system. There are taste buds on its upper section, which begins at the back of the mouth. The esophagus is surrounded at the top and bottom by two muscular rings, known respectively as the upper esophageal sphincter and the lower esophageal sphincter. Still without limiting the present invention in any way, these sphincters act to close the esophagus when food is not being swallowed. The upper esophageal sphincter surrounds the upper part of the esophagus and consists of skeletal muscle but is not under voluntary control. The lower esophageal sphincter, or gastroesophageal sphincter, surrounds the lower part of the esophagus at the junction between the esophagus and the stomach. It is also called the cardiac sphincter or cardio esophageal sphincter. The gastro-esophageal junction (also known as the esophagogastric junction) is the junction between the esophagus and the stomach, at the lower end of the esophagus. The transition from the esophageal mucosa to the gastric mucosa can be seen as an irregular zig-zag line, which is often called the z-line. Histological examination reveals abrupt transition between the stratified squamous epithelium of the esophagus and the simple columnar epithelium of the stomach. Normally, the cardia of the stomach is immediately distal to the z-line and the z-line coincides with the upper limit of the gastric folds of the cardia. However, when the anatomy of the mucosa is distorted in Barret's esophagus, the gastro-esophageal junction can be identified by the upper limit of the gastric folds rather than the mucosal transition. The functional location of the lower esophageal sphincter is generally situated about 3 cm below the z-line. The wall of the esophagus from the lumen outwards consists of mucosa, submucosa (connective tissue), layers of muscle fibers between layers of fibrous tissue, and an outer layer of connective tissue. The mucosa is a stratified squamous epithelium of around three layers of squamous cells, which contrasts to the single layer of columnar cells of the stomach. Most of the muscle is smooth muscle although striated muscle predominates in its upper third. Reference to “esophagus” should therefore be understood as a reference to any part of the structure described above.

Reference to “gastric” should be understood as a reference to the stomach, this being the muscular, hollow organ in the gastrointestinal tract. The stomach exhibits a dilated structure and functions as a vital digestive organ. It is located between the esophagus and the small intestine in the left upper part of the abdominal cavity. It secretes digestive enzymes and gastric acid to aid in food digestion. The pyloric sphincter controls the passage of partially digested food (chyme) from the stomach into the duodenum where peristalsis takes over to move this through the rest of the intestines. In humans, the stomach lies between the esophagus and the duodenum. A large double fold of visceral peritoneum, called the greater omentum, hangs down from the greater curvature of the stomach. Two sphincters keep the contents of the stomach contained; the lower esophageal sphincter (found in the cardiac region), at the junction of the esophagus and stomach, and the pyloric sphincter at the junction of the stomach with the duodenum.

The human stomach is divided into four sections, beginning at the cardia, each of which comprises different cells and functions.

The cardia is where the contents of the esophagus empty into the stomach.

The fundus is formed in the upper curved part.

The body is the main, central region of the stomach.

The pylorus is the lower section of the stomach that empties its contents into the duodenum.

The cardia is defined as the region following the “z-line” of the gastroesophageal junction, the point at which the epithelium changes from stratified squamous to columnar. Near the cardia is the lower esophageal sphincter. Without limiting the present invention in any way, the cardia is not an anatomically distinct region of the stomach but a region of the esophageal lining.

The human stomach walls consist of an outer mucosa, inner submucosa, muscularis externa, and serosa. The gastric mucosa of the stomach consists of the epithelium and the lamina propria (composed of loose connective tissue), with a thin layer of smooth muscle called the muscularis mucosae separating it from the submucosa beneath. The submucosa lies under the mucosa and consists of fibrous connective tissue, separating the mucosa from the next layer. Meissner's plexus occurs in this layer. The muscularis externa lies beneath the submucosa and is unique from other organs of the gastrointestinal tract, consisting of three layers:

The inner oblique layer: This layer is responsible for creating the motion that churns and physically breaks down the food. It is the only layer of the three which is not seen in other parts of the digestive system. The antrum has thicker skin cells in its walls and performs more forceful contractions than the fundus.

The middle circular layer: At this layer, the pylorus is surrounded by a thick circular muscular wall, which is normally tonically constricted, forming a functional pyloric sphincter, which controls the movement of chyme into the duodenum.

Auerbach's plexus (myenteric plexus) is found between the outer longitudinal and the middle circular layer and is responsible for the innervation of both.

The outer longitudinal layer is responsible for moving the bolus towards the pylorus of the stomach through muscular shortening.

The stomach also possesses a serosa, consisting of layers of connective tissue continuous with the peritoneum. In humans, different types of cells are found at the different layers of the gastric glands. The three types of gland are all located beneath the gastric pits within the gastric mucosa. The cardiac glands are found in the cardia of the stomach, enclosing the opening where the esophagus joins to the stomach. Only cardiac glands are found here and they primarily secrete mucus. They are fewer in number than the other gastric glands and are more shallowly positioned in the mucosa. The fundic glands, are found in the fundus and body of the stomach and the pyloric glands are located in the antrum of the pylorus. They secrete gastrin produced by their G cells. Reference to “gastric” should be understood as a reference to any of the regions described above.

Reference to “neoplasm” should be understood as a reference to a lesion, tumour or other encapsulated or unencapsulated mass or other form of growth which comprises neoplastic cells. A “neoplastic cell” should be understood as a reference to a cell exhibiting abnormal growth. The term “growth” should be understood in its broadest sense and includes reference to proliferation. In this regard, an example of abnormal cell growth is the uncontrolled proliferation of a cell. Another example is failed apoptosis in a cell, thus prolonging its usual life span. The neoplastic cell may be a benign cell or a malignant cell. Reference to benign neoplasms includes non-malignant neoplasms, pre-cancerous neoplasms or other pre-cancerous states. In a preferred embodiment, the subject neoplasm is an adenoma or an adenocarcinoma. To the extent that it is not expressly stipulated, reference to “adenoma” should be understood to also refer to dysplasia. Without limiting the present invention to any one theory or mode of action, an adenoma is generally a benign tumour of epithelial origin which is either derived from epithelial tissue or exhibits clearly defined epithelial structures. These structures may take on a glandular appearance and are typically characterized histologically by dysplasia. It can comprise a malignant cell population within the adenoma, such as occurs with the progression of a benign adenoma or benign neoplastic legion to a malignant adenocarcinoma.

Preferably, said neoplastic cell is an adenoma, dysplasia or adenocarcinoma and even more preferably an esophageal or gastric adenoma, dysplasia or adenocarcinoma.

Reference to “DNA region” should be understood as a reference to a specific section of genomic DNA. These DNA regions are specified either by reference to a gene name or a set of chromosomal coordinates. Both the gene names and the chromosomal coordinates would be well known to, and understood by, the person of skill in the art. As detailed hereinbefore, the chromosomal coordinates correspond to the Hg19 version of the genome. In general, a gene can be routinely identified by reference to its name, via which both its sequences and chromosomal location can be routinely obtained, or by reference to its chromosomal coordinates, via which both the gene name and its sequence can also be routinely obtained.

Reference to each of the genes/DNA regions detailed above should be understood as a reference to all forms of these molecules and to fragments or variants thereof. As would be appreciated by the person of skill in the art, some genes are known to exhibit allelic variation between individuals or single nucleotide polymorphisms. SNPs encompass insertions and deletions of varying size and simple sequence repeats, such as dinucleotide and trinucleotide repeats. Variants include nucleic acid sequences from the same region sharing at least 90%, 95%, 98%, or 99% sequence identity i.e. having one or more deletions, additions, substitutions, inverted sequences etc. relative to the DNA regions described herein. Accordingly, the present invention should be understood to extend to such variants which, in terms of the present diagnostic applications, achieve the same outcome despite the fact that minor genetic variations between the actual nucleic acid sequences may exist between individuals. The present invention should therefore be understood to extend to all forms of DNA, which arise from any other mutation, polymorphic or allelic variation.

It should be understood that the “individual” who is the subject of testing may be any human or non-human mammal. Examples of non-human mammals includes primates, livestock animals (e.g. horses, cattle, sheep, pigs, or donkeys), laboratory test animals (e.g. mice, rats, rabbits, or guinea pigs), companion animals (e.g. dogs, or cats) or captive wild animals (e.g. deer, or foxes).

Preferably the mammal is a human.

In accordance with these aspects and embodiments, in yet another embodiment said control level is a non-neoplastic level.

In one embodiment, said method is directed to screening either BCAT1 or IKZF1 in a biological sample.

In another embodiment, said method is directed screening both BCAT1 and IKZF1 in a biological sample.

In still another embodiment, said method is directed to screening both BCAT1 and IKZF1 but wherein only one of said genes exhibits a higher level of methylation.

In a further embodiment, said method is directed to screening both BCAT1 and IKZF1 but wherein both of said genes exhibits a higher level of methylation.

In still another embodiment, the neoplasm is malignant, such as an adenocarcinoma.

In a further embodiment, the neoplasm is non-malignant, such as an adenoma or dysplasia.

In terms of screening for the methylation of these gene regions, it should be understood that the assays can be designed to screen either the specific regions listed herein (which correspond to the “plus” strand of the gene) or the complementary “minus” strand. It is well within the skill of the person in the art to choose, which strand to analyse and to target that strand based on the chromosomal coordinates provided herein. In some circumstances, assays may be established to screen both strands.

It should be understood that one may screen for the specified panel of markers exclusively or one may elect to additionally screen for other markers, such as other DNA hypermethylation markers, other RNA expression level markers or other protein markers. These other markers may, for example, provide additional information in relation to the health status of the patient in issue.

Without limiting the present invention to any one theory or mode of action, although measuring the methylation levels across these DNA regions is diagnostic of an esophageal or gastric neoplastic condition, it has been determined that discrete subregions are particularly useful in this regard since these subregions contain a high density of CpG dinucleotides, which are frequently hypermethylated in esophageal or gastric neoplasias, such as esophageal or gastric cancers. This finding renders these subregions a particularly useful target for analysis since it both simplifies the screening process due to a shorter more clearly defined region of DNA requiring analysis and, further, the fact that the results from these regions will provide a significantly more definitive result in relation to the presence, or not, of hypermethylation than would be obtained if analysis was performed across the DNA region as a whole. This finding therefore both simplifies the screening process and increases the sensitivity and specificity of neoplasia diagnosis.

The subregions, which have been determined to exhibit particular utility, are listed below with reference to the gene and chromosomal region within which they are found:
(1) BCAT1 subregions chr12:25101992-25102093 (SEQ ID NO:1 or corresponding minus strand) and chr12:25101909-25101995 (SEQ ID NO:2 or corresponding minus strand); and
(2) IKZF1 subregions: chr7:50343867-50343961 (SEQ ID NO:3 or corresponding minus strand) and chr7:50343804-5033895 (SEQ ID NO:4 or corresponding minus strand).

Without limiting the present invention to any one theory or mode of action, the skilled person may screen one or more subregions for each gene marker.

In one embodiment, the methylation marker subregions tested for each selected gene marker are:
(1) The BCAT1 subregion defined by SEQ ID NO:1 or SEQ ID NO:2 or corresponding minus strand; and
(2) The IKZF1 subregion defined by SEQ ID NO:3 or SEQ ID NO:4 or corresponding minus strand;

Without limiting the present invention to any one theory or mode of action, DNA methylation is universal in bacteria, plants, and animals. DNA methylation is a type of chemical modification of DNA that is stable over rounds of cell division but does not involve changes in the underlying DNA sequence of the organism. Chromatin and DNA modifications are two important features of epigenetics and play a role in the process of cellular differentiation, allowing cells to stably maintain different characteristics despite containing the same genomic material. In eukaryotic organisms, DNA methylation occurs only at the number 5 carbon of the cytosine pyrimidine ring. In mammals, DNA methylation occurs mostly at the number 5 carbon of the cytosine of a CpG dinucleotide. CpG dinucleotides comprise approximately 1% human genome.

70-80% of all CpGs are methylated. CpGs may be grouped in clusters called “CpG islands” that are present in the 5′ regulatory regions of many genes and are frequently unmethylated. In many disease processes such as cancer, gene promoters and/or CpG islands acquire abnormal hypermethylation, which is associated with heritable transcriptional silencing. DNA methylation may impact the transcription of genes in two ways. First, the methylation of DNA may itself physically impede the binding of transcriptional proteins to the gene, thus blocking transcription. Second, methylated DNA may be bound by proteins known as Methyl-CpG-binding domain proteins (MBDs). MBD proteins then recruit additional proteins to the locus, such as histone deacetylases and other chromatin remodelling proteins that can modify histones, thereby forming compact, inactive chromatin termed silent chromatin. This link between DNA methylation and chromatin structure is very important. In particular, loss of Methyl-CpG-binding Protein 2 (MeCP2) has been implicated in Rett syndrome and Methyl-CpG binding domain protein 2 (MBD2) mediates the transcriptional silencing of hypermethylated genes in cancer.

In humans, the process of DNA methylation is carried out by three enzymes, DNA methyltransferase 1, 3a and 3b (DNMT1, DNMT3a, DNMT3b). It is thought that DNMT3a and DNMT3b are the de novo methyltransferases that set up DNA methylation patterns early in development. DNMT1 is the proposed maintenance methyltransferase that is responsible for copying DNA methylation patterns to the daughter strands during DNA replication. DNMT3L is a protein that is homologous to the other DNMT3s but has no catalytic activity. Instead, DNMT3L assists the de novo methyltransferases by increasing their ability to bind to DNA and stimulating their activity. Finally, DNMT2 has been identified as an “enigmatic” DNA methylstransferase homolog, containing all 10 sequence motifs common to all DNA methyltransferases; however, DNMT2 may not methylate DNA but instead has been shown to methylate a small RNA.

“Methylation status” should therefore be understood as a reference to the presence, absence and/or quantity or level of methylation at a particular nucleotide, or nucleotides, within a DNA region. The methylation status of a particular DNA sequence (e.g. DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, a methylation value, for example from the above described example, represents the methylation status and can thus be used as a quantitative indicator of the methylation status. This is of particular use when it is desirable to compare the methylation status of a sequence in a sample to a threshold value.

Methods of the present invention are predicated on the comparison of the level of methylation of specific DNA regions of a biological sample with the control methylation levels of these DNA regions. The “control level” may be the “normal level”, which is the level of methylation of the DNA region of a corresponding gastric or esophageal cell or cellular population which is not neoplastic or in another biological sample from which DNA may be isolated for assay.

The normal (or “non-neoplastic”) methylation level may be determined using non-neoplastic tissues derived from the same individual who is the subject of testing. However, it would be appreciated that this may be quite invasive for the individual concerned and it is therefore likely to be more convenient to analyse the test results relative to a standard result which reflects individual or collective results obtained from individuals other than the patient in issue. This latter form of analysis is in fact the preferred method of analysis since it enables the design of kits which require the collection and analysis of a single biological sample, being a test sample of interest. The standard results which provide the normal methylation level may be calculated by any suitable means which would be well known to the person of skill in the art. For example, a population of normal tissues can be assessed in terms of the level of methylation of the genes of the present invention, thereby providing a standard value or range of values against which all future test samples are analysed. It should also be understood that the normal level may be determined from the subjects of a specific cohort and for use with respect to test samples derived from that cohort. Accordingly, there may be determined a number of standard values or ranges which correspond to cohorts which differ in respect of characteristics such as age, gender, ethnicity or health status. Said “normal level” may be a discrete level or a range of levels. An increase in the methylation level of the subject genes relative to normal levels is indicative of the tissue being neoplastic.

The term “methylation” shall be taken to mean the presence of a methyl group added by the action of a DNA methyl transferase enzyme to a cytosine base or bases in a region of nucleic acid, e.g. genomic DNA. As described herein, there are several methods known to those skilled in the art for determining the level or degree of methylation of nucleic acid.

By “higher level” is meant that there are a higher number of methylated CpG dinucleotides in the subject diagnosed than in a control sample, that is, either the proportion of DNA molecules methylated at a particular CpG site is higher or there are a higher number of separate CpG sites methylated in the subject. It should be understood that the terms “enhanced” and “increased” are used interchangeably with the term “higher”. A higher number of methylated CpG dinucleotides in a diagnosed subject may be 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 96%, 97%, 98%, 99%, 100%, 200%, 300%, 400%, or 500% or any number within a range defined by any two of the aforementioned percentages greater than in a control sample.

In relation to detecting a “higher level” of methylation, it should be understood that in some situations the normal level will in fact correspond to the absence of any detectable methylation while the neoplastic level will correspond to the presence of methylation, per se. In this situation the diagnostic method is relatively simple since one need only screen for the mere presence of methylation (e.g., a qualitative assessment only), rather than assessing the methylation levels relative to a control level of methylation, which analysis necessarily involves a measure of quantification. Without limiting the present invention in any way, it is observed in blood-derived samples, for example, that in the context of some markers the change in methylation of that marker upon the onset of neoplasia is a shift from undetectable levels of methylation to the presence of detectable methylation. In these situations, a relatively simple qualitative assessment is enabled where one need only screen a test sample to determine the presence or not of methylation. In the context of the definitions provided herein, reference to “higher level” encompasses both a relative increase in the level of methylation of a marker or the onset of methylation where previously none was evident. As detailed hereinbefore, the control level may be newly assessed for each patient or there may be a standard result against which all test samples are assessed. Where it is known that methylation is not present on the marker of interest, one need only screen for the presence or not of methylation since the control level is the absence of methylation and the “higher level” is thereby the presence of any amount of methylation.

It should also be understood that the control level may be the level of methylation of a previously tested biological sample from the same tested individual or from another individual (e.g., a healthy subject or a subject having cancer). As discussed in more detail hereafter, this is particularly useful in the context of monitoring a patient for recurrence, therapeutic efficacy or the like.

The present invention is not to be limited by a precise number of methylated residues that are considered to be diagnostic of neoplasia in a subject, because some variation between patient samples will occur. The present invention is also not limited by positioning of the methylated residue.

Nevertheless, a number of specific cytosine residues, which undergo hypermethylation within these subregions have also been identified. In another embodiment, therefore, a screening method can be employed, which is specifically directed to assessing the methylation status of one or more of either these residues or the corresponding cytosine at position n+1 on the opposite DNA strand.

It should be appreciated by the person of skill in the art that these individual residues are numbered by reference to Hg19, which also corresponds to the numbering of the specific subregions listed hereinbefore and which can be further identified when the coordinate numbering for each subregion is applied to the corresponding subregion sequences which are provided in the sequence listing. It should be understood that these residues have been identified in the context of the subregion DNA. However, there are other residues which are hypermethylated outside the subregions themselves but within the larger DNA region from which the subregions derive. Accordingly, these specified residues represent a particularly useful subset of individual cytosine residues which undergo hypermethylation within the context of the DNA regions and subregions herein disclosed. These individual residues are grouped below according to the DNA region within which they occur. These DNA regions are identified by reference to both the Hg19 chromosomal coordinates and the gene region name.

To the extent that the method of the present invention includes analysing IKZF1, the subject residues are:

| | | |
|---|---|---|
| chr7: 50343869 | chr7: 50343872 | chr7: 50343883 |
| chr7: 50343889 | chr7: 50343890 | chr7: 50343897 |
| chr7: 50343907 | chr7: 50343909 | chr7: 50343914 |
| chr7: 50343934 | chr7: 50343939 | chr7: 50343950 |
| chr7: 50343959 | chr7: 50343805 | chr7: 50343822 |
| chr7: 50343824 | chr7: 50343826 | chr7: 50343829 |
| chr7: 50343831 | chr7: 50343833 | chr7: 50343838 |
| chr7: 50343847 | chr7: 50343850 | chr7: 50343858 |
| chr7: 50343864 | chr7: 50343869 | chr7: 50343872 |
| chr7: 50343890 | | |

or a corresponding cytosine at position n+1 on the opposite DNA strand.

The detection methods of the present invention can be performed on any suitable biological sample. To this end, reference to a “biological sample” should be understood as a reference to any sample of biological material derived from an animal such as, but not limited to, cellular material, biofluids (e.g. blood), tissue biopsy specimens, saliva, urine, surgical specimens or fluid which has been introduced into the body of an animal and subsequently removed. The biological sample which is tested according to the method of the present invention may be tested directly or may require some form of treatment prior to testing. For example, a biopsy or surgical sample may require homogenisation prior to testing or it may require sectioning for in situ testing of the qualitative expression levels of individual genes. Alternatively, a cell sample may require permeabilisation prior to testing. Further, to the extent that the biological sample is not in liquid form, (if such form is required for testing) it may require the addition of a reagent, such as a buffer, to mobilise the sample.

To the extent that the DNA region of interest is present in a biological sample, the biological sample may be directly tested or else all or some of the nucleic acid present in the biological sample may be isolated prior to testing. In yet another example, the sample may be partially purified or otherwise enriched prior to analysis. For example, to the extent that a biological sample comprises a very diverse cell population, it may be desirable to enrich for a sub-population of particular interest. It is within the scope of the present invention for the target cell population or molecules derived therefrom to be treated prior to testing, for example, inactivation of live virus. It should also be understood that the biological sample may be freshly harvested or it may have been stored (for example by freezing) prior to testing or otherwise treated prior to testing (such as by undergoing culturing).

The choice of what type of sample is most suitable for testing in accordance with the method disclosed herein will be dependent on the nature of the situation. Preferably, said sample is a surgical resection, tissue biopsy or blood sample (e.g. whole blood, serum, plasma, exosomes, or buffy coat). Reference to “buffy coat” should be understood as a reference to the fraction of an anticoagulated blood sample that contains most of the white blood clles and platelets following density gradient centrifugation of the blood.

Reference to “exosomes” as used herein should be understood as membrane bound extracellular vesicles about 10 to 100 nm that are released from cells during the fusion of the plasma membrane with endosomal multivesicular bodies containing intraluminal vesicles. These exosomes carry biological molecules including but not limited to lipids, proteins, RNA, DNA, cholesterol, sphingolipids, ceramides, fatty acids, phospholipids, surface markers, antigen presenting proteins, major histocompatibility complexes, adhesins, immunosuppressive proteins, cytoskeleton proteins, trafficking proteins, messenger RNA (mRNA), microRNA (miRNA), long noncoding RNA (lncRNA), double stranded DNA (dsDNA), genomic DNA (gDNA), exosomal DNA, methylated DNA, or any combination thereof, that are sourced from the cytosol of the originating cell, and can cause downstream effects on other cells through these components. Exosomes have been implicated in many cancer proliferation processes, such as invasion to nearby tissue, establishing pre-metastatic niches, migration, metastasis, immune modulation, and angiogenesis. Exosomes from both normal and diseased cells can be found in biological fluids such as blood, urine, lymph, and cerebrospinal fluid. Consequently, non-invasive or minimally invasive diagnostics using exosomes can help determine the onset or predisposition of the onset of diseases such as cancer. Exosomes can be purified from said biological fluids, or from in vitro cell culture medium, using techniques known in the art, including but not limited to centrifugation, ultracentrifugation, density gradient ultracentrifugation, filtration, ultrafiltration, chromatography, size exclusion chromatography, affinity chromatography, precipitation, or microfluidics, or any combination thereof. Purified exosomes can be processed using techniques known in the art, such as to extract the constituent lipids, proteins, RNA or DNA for subsequent analyses including but not limited to polymerase chain reaction (PCR), quantitative PCR (qPCR), reverse transcription PCR (rtPCR), mass spectrometry, chromatography, electrophoresis, sequencing, or to be added to other in vivo or in vitro biological systems to induce an effect.

More preferably, said biological sample is a blood sample or biopsy sample.

Still more preferably, said method is directed to screening the methylation of plasma derived cell-free DNA, such as circulating tumor DNA.

As detailed hereinbefore, the present invention is designed to screen for a neoplastic cell or cellular population, which is located in the esophagus or stomach. Accordingly, reference to “cell or cellular population” should be understood as a reference to an individual cell or a group of cells. Said group of cells may be a diffuse population of cells, a cell suspension, an encapsulated population of cells or a population of cells which take the form of tissue.

Reference to the “onset” of a neoplasm, such as adenoma or adenocarcinoma, should be understood as a reference to one or more cells of that individual exhibiting dysplasia. In this regard, the adenoma or adenocarcinoma may be well developed in that a mass of dysplastic cells has developed. Alternatively, the adenoma or adenocarcinoma may be at a very early stage in that only relatively few abnormal cell divisions have occurred at the time of diagnosis. In some embodiments, dysplasia is preceded by metaplasia or other predysplastic stages. In some embodiments, oesophagal dysplasia may be preceded by the development of Barrett's columnar-lined oesophageal mucosa, which may further progress to resemble intestinal metaplasia. In some embodiments, cells of these predysplastic stages may exhibit changed methylation levels. The present invention also extends to the assessment of an individual's predisposition to the development of a neoplasm, such as an adenoma or adenocarcinoma. Without limiting the present invention in any way, changed methylation levels, as compared to a control, may be indicative of that individual's predisposition to developing a neoplasia, such as the future development of an adenoma or adenocarcinoma or another adenoma or adenocarcinoma.

Although the preferred method is to assess methylation levels for the purpose of diagnosing neoplasia development or predisposition thereto, the detection of converse changes in the levels of said methylation may be desired under certain circumstances, for example, to monitor the effectiveness of therapeutic or prophylactic treatment directed to modulating a neoplastic condition, such as adenoma or adenocarcinoma development. For example, where elevated levels of methylation indicate that an individual has developed a condition characterised by adenoma or adenocarcinoma development, screening for a decrease in the levels of methylation subsequently to the onset of a therapeutic treatment regime may be utilised to indicate successful clearance of the neoplastic cells. In another example, one can use this method to test the tissue at the margins of a tumour resection in order to determine whether the full margin of the tumour has been removed.

In some embodiments, the present methods can therefore be used in the diagnosis, prognosis, classification, prediction of disease risk, detection of recurrence of disease, selection of treatment, or monitoring of a number of types of predysplasias, dysplasias, preneoplasias, and neoplasias. In some embodiments, a cancer at any stage of progression can be detected, including but not limited to predysplastic, dysplastic, preneoplastic, neoplastic, non-neoplastic, primary, metastatic, or recurrent cancers.

The present invention provides methods for determining whether a mammal (e.g., a human) has a neoplasia of the esophagus or stomach, whether a biological sample taken from a mammal contains neoplastic cells or DNA derived from neoplastic cells, estimating the risk or likelihood of a mammal developing a neoplasm, monitoring the efficacy of anti-cancer treatment, or selecting the appropriate anti-cancer treatment in a mammal with cancer. Such methods are based on the determination that neoplastic cells have a different methylation status than normal cells in the DNA regions described herein. Accordingly, by determining whether or not a cell contains differentially methylated sequences in the DNA regions as described herein, it is possible to determine that a cell is neoplastic.

The methods of the invention can be used to evaluate individuals known or suspected to have a neoplasia or as a routine clinical test, e.g., in an individual not necessarily suspected to have a neoplasia. Further diagnostic assays or tests can be performed to confirm the type and status of neoplasia in the individual, such as endoscopy.

Further, the present methods may be used to assess the efficacy of a course of treatment. For example, the efficacy of an anti-cancer treatment can be assessed by monitoring DNA methylation of the sequences described herein over time in a mammal having cancer. For example, a reduction or absence of methylation in any of the diagnostic sequences of the invention in a biological sample taken from a mammal following a treatment, compared to a level in a sample taken from the mammal before, or earlier in, the treatment, indicates efficacious treatment.

The methods of the present invention are therefore useful as a one-time test or as an on-going monitor of those individuals thought to be at risk of neoplasia development or as a monitor of the effectiveness of therapeutic or prophylactic treatment regimes directed to inhibiting or otherwise slowing neoplasia development. In these situations, mapping the modulation of methylation levels in any one or more classes of biological samples is a valuable indicator of the status of an individual or the effectiveness of a therapeutic or prophylactic regime, which is currently in use. Accordingly, the method of the present invention should be understood to extend to monitoring for increases or decreases in methylation levels in an individual relative to their normal level (as hereinbefore defined), or relative to one or more earlier methylation levels determined from a biological sample of said individual.

The methods for detecting neoplasia can comprise the detection of one or more other cancer-associated polynucleotide or polypeptides sequences. Accordingly, detection of methylation by the method of the invention can be used either alone, or in combination with other screening methods for the diagnosis or prognosis of neoplasia.

Any method for detecting DNA methylation can be used in the methods of described herein. A number of methods are available for detection of differentially methylated DNA at specific loci in either primary tissue samples or in patient samples such as blood, urine, stool or saliva (reviewed in Kristensen and Hansen Clin Chem. 55:1471-83, 2009; Ammerpohl et al. Biochim Biophys Acta. 1790:847-62, 2009; Shames et al. Cancer Lett. 251:187-98, 2007; Clark et al. Nat Protoc. 1:2353-64, 2006). For analysis of the proportion or extent of DNA methylation in a target gene, DNA is normally treated with sodium bisulfite and regions of interest amplified using primers and PCR conditions that will amplify independently of the methylation status of the DNA. The methylation of the overall amplicon or individual CpG sites can then be assessed by sequencing, including pyrosequencing, restriction enzyme digestion (COBRA) or by melting curve analysis. Alternatively, ligation-based methods for analysis of methylation at specific CpG sites may be used. Detection of aberrantly methylated DNA released from tumours and into bodily fluids is being developed as a means of cancer diagnosis. Here, in the case of hypermethylated sequences, it is necessary to use sensitive methods that allow the selective amplification of the methylated DNA sequence from a background of normal cellular DNA that is unmethylated. Such methods based on bisulfite-treated DNA, for example; include methylation selective PCR (MSP), Heavymethyl PCR, Headloop PCR or Helper-dependent chain reaction (PCT/AU2008/001475).

Briefly, in some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. patent application Ser. Nos. 10/971,986; 11/071,013; and Ser. No. 10/971,339. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (e.g., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. A methylation-sensitive enzyme is one, which cuts DNA if its recognition sequence is unmethylated while a methylation-dependent enzyme cuts DNA if its recognition sequence is methylated. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. patent application Ser. No. 10/971,986.

Kits for the above methods can include, e.g., one or more of methylation-dependent restriction enzymes, methylation-sensitive restriction enzymes, amplification (e.g., PCR) reagents, probes and/or primers.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol. Biotechnol. 20(2):163-79 (2002). Amplifications may be monitored in “real time.”

Additional methods for detecting DNA methylation can involve genomic sequencing before and after treatment of the DNA with bisulfite. See, e.g., Frommer et al., Proc. Natl. Acad. Sci. USA 89:1827-1831 (1992). When sodium bisulfite is contacted to DNA, unmethylated cytosine is converted to uracil, while methylated cytosine is not modified.

In some embodiments, restriction enzyme digestion of PCR products amplified from bisulfite-converted DNA is used to detect DNA methylation. See, e.g., Sadri & Hornsby, Nucl. Acids Res. 24:5058-5059 (1996); Xiong & Laird, Nucleic Acids Res. 25:2532-2534 (1997).

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated verses unmethylated DNA. See, Herman et al. Proc. Natl. Acad. Sci. USA 93:9821-9826 (1996); U.S. Pat. No. 5,786,146.

In some embodiments, a MethyLight assay is used alone or in combination with other methods to detect DNA methylation (see, Eads et al., Cancer Res. 59:2302-2306 (1999)). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of methylated DNA, (or alternatively to unmethylated sequences) amplification can indicate methylation status of sequences where the primers hybridize. Furthermore, the amplification product can be detected with a probe that specifically binds to a sequence resulting from bisulfite treatment of a unmethylated DNA. If desired, both primers and probes can be used to detect methylation status. Thus, kits for use with MethyLight can include sodium bisulfite as well as primers or detectably-labelled probes (including but not limited to Tagman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect DNA methylation (see, Gonzalgo & Jones, Nucleic Acids Res. 25:2529-2531 (1997)). The Ms-SNuPE technique is a quantitative method for assessing methylation differences at specific CpG sites based on bisulfite treatment of DNA, followed by single-nucleotide primer extension (Gonzalgo & Jones, supra). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers, deoxynucleotides; gel extraction kit; positive control primers; Ms-SNuPE primers for a specific gene; reaction buffer (for the Ms-SNuPE reaction); or detectably-labelled nucleotides or any combination thereof. Additionally, bisulfite conversion reagents may include: DNA denaturation buffer; sulfonation buffer; DNA recovery regents or kit (e.g., precipitation, ultrafiltration, affinity column); desulfonation buffer; or DNA recovery components.

Additional methylation detection methods include, but are not limited to, methylated CpG island amplification (see, Toyota et al., Cancer Res. 59:2307-12 (1999)) and those described in, e.g., U.S. Patent Publication 2005/0069879; Rein, et al. Nucleic Acids Res. 26 (10): 2255-64 (1998); Olek, et al. Nat. Genet. 17(3): 275-6 (1997); and PCT Publication No. WO 00/70090.

More detailed information in relation to several of these generally described methods is provided below:

(a) Probe or Primer Design and/or Production

Several methods described herein for the diagnosis of a neoplasia use one or more probes and/or primers. Methods for designing probes and/or primers for use in, for example, PCR or hybridization are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design optimal probes and/or primers for a variety of assays, e.g. Primer 3 available from the Center for Genome Research, Cambridge, Mass., USA.

Clearly, the potential use of the probe or primer should be considered during its design. For example, should the probe or primer be produced for use in a methylation specific PCR or ligase chain reaction (LCR) assay, the nucleotide at the 3′ end (or 5′ end in the case of LCR) should preferably correspond to a methylated nucleotide in a nucleic acid.

Probes and/or primers useful for detection of a sequence associated with a neoplasia are assessed, for example, to determine those that do not form hairpins, self-prime or form primer dimers (e.g. with another probe or primer used in a detection assay). Furthermore, a probe or primer (or the sequence thereof) is often assessed to determine the temperature at which it denatures from a target nucleic acid (e.g., the melting temperature of the probe or primer, or Tm). Methods for estimating Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to 100 nucleotides or about 100 nucleotides) chemical synthesis is preferable.

For longer sequences standard replication methods employed in molecular biology are useful, such as, for example, the use of M13 for single stranded DNA as described by Messing, Methods Enzymol, 101, 20-78, 1983. Other methods for oligonucleotide synthesis include, for example, phosphotriester and phosphodiester methods (Narang, et al. Meth. Enzymol 68: 90, 1979) and synthesis on a support (Beaucage, et al. Tetrahedron Letters 22:1859-1862, 1981) as well as phosphoramidate technique, Caruthers, M. H., et al., Methods in Enzymology, Vol. 154, pp. 287-314 (1988), and others described in “Synthesis and Applications of DNA and RNA,” S. A. Narang, editor, Academic Press, New York, 1987, and the references cited therein. Probes comprising locked nucleic acid (LNA) are synthesized as described, for example, in Nielsen et al. J. Chem. Soc. Perkin Trans., 1:3423, 1997; Singh and Wengel, Chem. Commun. 1247, 1998., while probes comprising peptide-nucleic acid (PNA) are synthesized as described, for example, in Egholm et al., Am. Chem. Soc., 114:1895, 1992; Egholm et al., Nature, 365:566, 1993; and Orum et al., Nucl. Acids Res., 21:5332, 1993.

The primers, probes, or other nucleic acids described herein comprise nucleobases. Primary, canonical, natural, or unmodified bases are adenine (A), cytosine (C), guanine (G), thymine (T), and uracil (U). In some embodiments, it may be preferable to design a primer, probe, or other nucleic acid with multiple possible alternative nucleobase at a specific location (degenerate base), for example, to increase annealing of a primer or probe to a specific nucleic acid, a population of nucleic acid alleles, or a population of bisulfite-treated and untreated nucleic acids. Degenerate bases are represented according to an International Union of Pure and Applied Chemistry (IUPAC) standard. These representations include but are not limited to W (A and T), S (C and G), M (A and C), K (G and T), R (A and G), Y (C and T), B (C, G, and T), D (A, G, and T), H (A, C, and T), V (A, C, and G), or N (A, C, G, and T). Other “non-canonical” nucleobases include but are not limited to purines, pyrimidines, modified nucleobases, 5-methylcytosine, pseudouridine, dihydrouridine, 2,6-diaminopurine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

### (b) Methylation-Sensitive Endonuclease Digestion of DNA

In one example, the increased methylation in a sample is determined using a process comprising contacting the nucleic acid with an amount of a methylation-sensitive restriction endonuclease enzyme under conditions sufficient for the nucleic acid to be digested and then detecting the fragments produced. Exemplary methylation-sensitive endonucleases include, for example, HhaI or HpaII. Preferably, assays include internal controls that are digested with a methylation-insensitive enzyme having the same specificity as the methylation-sensitive enzyme employed. For example, the methylation-insensitive enzyme MspI is an isoschizomer of the methylation-sensitive enzyme HpaII.

### Hybridization Assay Formats

In one example, the digestion of a nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization (Kawai et al., Mol. Cell. Biol. 14:7421-7427, 1994; Gonzalgo et al., Cancer Res. 57:594-599, 1997).

Suitable hybridization conditions are determined based on the melting temperature (Tm) of a nucleic acid duplex comprising the probe. The skilled artisan will be aware that optimum hybridization reaction conditions should be determined empirically for each probe, although some generalities can be applied. Preferably, hybridizations employing short oligonucleotide probes are performed at low to medium stringency. In the case of a GC rich probe or primer or a longer probe or primer a high stringency hybridization and/or wash is preferred. A high stringency is defined herein as being a hybridization and/or wash carried out in 0.1×SSC buffer or about 0.1×SSC buffer and/or 0.1% (w/v) SDS or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

In accordance with the examples, a difference in the fragments produced for the test sample and a negative control sample is indicative of the subject having a neoplasia. Similarly, in cases where the control sample comprises data from a tumor, cancer tissue or a cancerous cell or pre-cancerous cell, similarity, albeit not necessarily absolute identity, between the test sample and the control sample is indicative of a positive diagnosis (e.g., cancer). Once it is determined that the sample has a methylation level that differs from the control, the subject can be provided or guided to or advised to receive a therapy or treatment protocol, which aligns with the diagnostic result obtained (e.g., colonoscopy, surgical removal of malignant tissue, and/or radiation, chemotherapy, or immunotherapy).

### Amplification Assay Formats

In an alternative, the fragments produced by the restriction enzyme are detected using an amplification system, such as, for example, polymerase chain reaction (PCR), rolling circle amplification (RCA), inverse polymerase chain reaction (iPCR), in situ PCR (Singer-Sam et al., Nucl. Acids Res. 18:687, 1990), strand displacement amplification (SDA) or cycling probe technology.

Methods of PCR are known in the art and described, for example, by McPherson et al., PCR: A Practical Approach. (series eds, D. Rickwood and B. D. Hames), IRL Press Limited, Oxford. pp 1-253, 1991 and by Dieffenbach (ed) and Dveksler (ed) (In: PCR Primer: A Laboratory Manual, Cold Spring Harbour Laboratories, NY, 1995), the contents of which are each incorporated in their entirety by way of reference. Generally, for PCR, two non-complementary nucleic acid primer molecules comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides are hybridized to different strands of a nucleic acid template molecule at their respective annealing sites, and specific nucleic acid molecule copies of the template that intervene the annealing sites are amplified enzymatically. In some embodiments, the two non-complementary nucleic acid primer molecules are at least 18 nucleotides or at least about 18 nucleotides in length. In some embodiments, the two non-complementary nucleic acid primer molecules are at least 20-30 nucleotides or at least about 20-30 nucleotides in length. Amplification products may be detected, for example, using electrophoresis and detection with a detectable marker that binds nucleic acids. Alternatively, one or more of the oligonucleotides are labelled with a detectable marker (e.g. a fluorophore) and the amplification product detected using, for example, a lightcycler (Perkin Elmer, Wellesley, Mass., USA, Roche Applied Science, Indianapolis, IN, USA).

Strand displacement amplification (SDA) utilizes oligonucleotide primers, a DNA polymerase and a restriction endonuclease to amplify a target sequence. The oligonucleotides are hybridized to a target nucleic acid and the polymerase is used to produce a copy of the region intervening the primer annealing sites. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Cycling Probe Technology uses a chimeric synthetic primer that comprises DNA-RNA-DNA that is capable of hybridizing to a target sequence. Upon hybridization to a target sequence the RNA-DNA duplex formed is a target for RNaseH thereby cleaving the primer. The cleaved primer is then detected, for example, using mass spectrometry or electrophoresis.

For primers that flank or are adjacent to a methylation-sensitive endonuclease recognition site, it is preferred that such primers flank only those sites that are hypermethylated in neoplasia to ensure that a diagnostic amplification product is produced. In this regard, an amplification product will only be produced when the restriction site is not cleaved, i.e., when it is methylated. Accordingly, detection of an amplification product indicates that the CpG dinucleotide/s of interest is/are methylated.

As will be known to the skilled artisan, the precise length of the amplified product will vary depending upon the distance between the primers. Clearly this form of analysis may be used to determine the methylation status of a plurality of CpG dinucleotides provided that each dinucleotide is within a methylation sensitive restriction endonuclease site. In these methods, one or more of the primers may be labelled with a detectable marker to facilitate rapid detection of amplified nucleic acid, for example, a fluorescent label (e.g. Cy5 or Cy3) or a radioisotope (e.g. ³²P).

The amplified nucleic acids are generally analyzed using, for example, non-denaturing agarose gel electrophoresis, non-denaturing polyacrylamide gel electrophoresis, mass spectrometry, liquid chromatography (e.g. HPLC or dHPLC), or capillary electrophoresis. (e.g. MALDI-TOF). High throughput detection methods, such as, for example, matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or DNA chip technology (e.g., WO98/49557; WO 96/17958; Fodor et al., Science 767-773, 1991; U.S. Pat. Nos. 5,143,854; and 5,837,832, the contents of which are all incorporated herein by reference), are especially preferred for all assay formats described herein. Alternatively, amplification of a nucleic acid may be continuously monitored using a melting curve analysis method as described herein and/or in, for example, U.S. Pat. No. 6,174,670, which is incorporated herein by reference.

### (c) Other Assay Formats

In an alternative example, the increased methylation in a sample is determined by performing a process comprising treating chromatin containing the nucleic acid with an amount of DNaseI under conditions sufficient for nucleic acid to be digested and then detecting the fragments produced. This assay format is predicated on the understanding that chromatin containing methylated DNA, e.g., hyper methylated DNA, has a more tightly-closed conformation than non-hyper methylated DNA and, as a consequence, is less susceptible to endonuclease digestion by DNase I.

In accordance with this method, DNA fragments of different lengths are produced by DNase I digestion of methylated compared to non-methylated DNA. Such different DNA fragments are detected, for example, using an assay described earlier. Alternatively, the DNA fragments are detected using PCR-SSCP essentially as described, for example, in Gregory and Feil, Nucleic Acids Res., 27, e32i-e32iv, 1999. In adapting PCR-SSCP to the present invention, amplification primers flanking or comprising one or more CpG dinucleotides in a nucleic acid that are resistant to DNase I digestion in a neoplasia sample but not resistant to DNase I digestion in a healthy/normal control or healthy/normal test sample are used to amplify the DNase I-generated fragments. In this case, the production of a specific nucleic acid fragment using DNase I is diagnostic of neoplasia, because the DNA is not efficiently degraded. In contrast, template DNA from a healthy/normal subject sample is degraded by the action of DNase I and, as a consequence, amplification fails to produce a discrete amplification product. Alternative methods to PCR-SSCP, such as for example, PCR-dHlPLC are also known in the art and contemplated by the present invention.

### (d) Selective Mutagenesis of Non-Methylated DNA

In an alternative method the increased methylation in a sample is determined using a process comprising contacting the nucleic acid with an amount of a compound that selectively mutates a non-methylated cytosine residue within a CpG dinucleotide under conditions sufficient to induce mutagenesis.

Preferred compounds mutate cytosine to uracil or thymidine, such as, for example, a salt of bisulfite, e.g., sodium bisulfite or potassium bisulfite (Frommer et al., 1992, supra). Bisulfite treatment of DNA is known to distinguish methylated from non-methylated cytosine residues, by mutating cytosine residues that are not protected by methylation, including cytosine residues that are not within a CpG dinucleotide or that are positioned within a CpG dinucleotide that is not subject to methylation.

### Sequence Based Detection

In one example, the presence of one or more mutated nucleotides or the number of mutated sequences is determined by sequencing mutated DNA. One form of analysis comprises amplifying mutated nucleic acid using an amplification reaction described herein, for example, PCR. The amplified product is then directly sequenced or cloned and the cloned product sequenced. Methods for sequencing DNA are known in the art and include for example, the dideoxy chain termination method or the Maxam-Gilbert method (see Sambrook et al., Molecular Cloning, A Laboratory Manual (2nd Ed., CSHP, New York 1989) or Zyskind et al., Recombinant DNA Laboratory Manual, (Acad. Press, 1988)).

As the contact of a nucleic acid with a compound, such as, for example, bisulfite results in non-methylated cytosines being mutated to uracil (and hence thymidine after an amplification process), analysis of the sequence determines the presence or absence of a methylated nucleotide. For example, by comparing the sequence obtained using a control sample or a sample that has not been treated with bisulfite, or the known nucleotide sequence of the region of interest with a contacted sample facilitates the detection of differences in the nucleotide sequence. Any thymine residue detected at the site of a cytosine in the contacted sample compared to a control or untreated sample may be considered to be caused by mutation as a result of bisulfite contact. Suitable methods for the detection of methylation using sequencing of bisulfite treated nucleic acid are described, for example, in Frommer et al., 1992, supra or Clark et al., Nucl. Acids Res. 22:2990-2997, 1994.

In another method, the presence of a mutated or non-mutated nucleotide in a bisulfite treated sample is detected using pyrosequencing, such as, for example, as described in Uhlmann et al., Electrophoresis, 23: 4072-4079, 2002. Essentially this method is a form of real-time sequencing that uses a primer that hybridizes to a site adjacent or close to the site of a cytosine that is methylated. Following hybridization of the primer and template in the presence of a DNA polymerase each of four modified deoxynucleotide triphosphates are added separately according to a predetermined dispensation order. Only an added nucleotide that is complementary to the bisulfite treated sample is incorporated and inorganic pyrophosphate (PPi) is liberated. The PPi then drives a reaction resulting in production of detectable levels of light. Such a method allows determination of the identity of a specific nucleotide adjacent to the site of hybridization of the primer.

Methods of solid phase pyrosequencing are known in the art and reviewed in, for example, Landegren et al., Genome Res., 8(8): 769-776, 1998. Such methods enable the high-throughput detection of methylation of a number of CpG dinucleotides.

A related method for determining the sequence of a bisulfite contacted nucleotide is methylation-sensitive single nucleotide primer extension (Me-SnuPE) or SNaPmeth. Suitable methods are described, for example, in Gonzalgo and Jones, 1997, supra, or Uhlmann et al., Electrophoresis, 23:4072-4079, 2002. An oligonucleotide is used that hybridizes to the region of a nucleic acid adjacent to the site of a cytosine that is methylated. This oligonucleotide is then used in a primer extension protocol with a polymerase and a free nucleotide diphosphate or dideoxynucleotide triphosphate that corresponds to either or any of the possible bases that occur at this site following bisulfite treatment (i.e., thymine or cytosine). Preferably, the nucleotide-diphosphate is labelled with a detectable marker (e.g. a fluorophore). Following primer extension, unbound labelled nucleotide diphosphates are removed, e.g. using size exclusion chromatography or electrophoresis, or hydrolyzed, using for example, alkaline phosphatase, and the incorporation of the labelled nucleotide to the oligonucleotide is detected, indicating the base that is present at the site.

Other high throughput sequencing methods are encompassed by the present invention. Such methods include, for example, solid phase minisequencing (as described, for example, in Southern et al., Genomics, 13:1008-1017, 1992), or minisequencing with FRET (as described, for example, in Chen and Kwok, Nucleic Acids Res. 25:347-353, 1997).

### Restriction Endonuclease-Based Assay Format

In one method, the presence of a non-mutated sequence is detected using combined bisulfite restriction analysis (COBRA) essentially as described in Xiong and Laird, 2001, supra. This method exploits the differences in restriction enzyme recognition sites between methylated and unmethylated nucleic acid after contact with a compound that selectively mutates a non-methylated cytosine residue, e.g., bisulfite.

Following bisulfite treatment, a region of interest comprising one or more CpG dinucleotides that are methylated and are included in a restriction endonuclease recognition sequence is amplified using an amplification reaction described herein, e.g., PCR. The amplified product is then contacted with the restriction enzyme that cleaves at the site of the CpG dinucleotide for a time and under conditions sufficient for cleavage to occur. A restriction site may be selected to indicate the presence or absence of methylation. For example, the restriction endonuclease TaqI cleaves the sequence TCGA, following bisulfite treatment of a non-methylated nucleic acid the sequence will be TTGA and, as a consequence, will not be cleaved. The digested and/or non-digested nucleic acid is then detected using a detection means known in the art, such as, for example, electrophoresis and/or mass spectrometry. The cleavage or non-cleavage of the nucleic acid is indicative of cancer in a subject. Clearly, this method may be employed in either a positive read-out or negative read-out system for the diagnosis of a cancer.

### Positive Read-Out Assay Format

In one embodiment, the assay format comprises a positive read-out system in which DNA from a sample that has been treated, for example, with bisulfite is detected as a positive signal. Preferably, the non-hypermethylated DNA from a healthy or normal control subject is not detected or only weakly detected.

In a preferred embodiment, the increased methylation in a subject sample is determined using a process comprising:
(i) contacting the nucleic acid with an amount of a compound that selectively mutates a non-methylated cytosine residue under conditions sufficient to induce mutagenesis thereby producing a mutated nucleic acid;
(ii) hybridizing a nucleic acid to a probe or primer comprising a nucleotide sequence that is complementary to a sequence comprising a methylated cytosine residue under conditions such that selective hybridization to the non-mutated nucleic acid occurs; and

(iii) detecting the selective hybridization.

In this context, the term “selective hybridization” means that hybridization of a probe or primer to the non-mutated nucleic acid occurs at a higher frequency or rate, or has a higher maximum reaction velocity, than hybridization of the same probe or primer to the corresponding mutated sequence. Preferably, the probe or primer does not hybridize to the non-methylated sequence carrying the mutation(s) under the reaction conditions used.

### Hybridization-Based Assay Format

In one embodiment, the hybridization is detected using Southern, dot blot, slot blot or other nucleic acid hybridization means (Kawai et al., 1994, supra; Gonzalgo et al., 1997, supra). Subject to appropriate probe selection, such assay formats are generally described herein above and apply mutatis mutandis to the presently described selective mutagenesis approach.

Preferably, a ligase chain reaction format is employed to distinguish between a mutated and non-mutated nucleic acid. Ligase chain reaction (described in EP 320,308 and U.S. Pat. No. 4,883,750) uses at least two oligonucleotide probes that anneal to a target nucleic acid in such a way that they are juxtaposed on the target nucleic acid. In a ligase chain reaction assay, the target nucleic acid is hybridized to a first probe that is complementary to a diagnostic portion of the target sequence (the diagnostic probe) e.g., a nucleic acid comprising one or more methylated CpG dinucleotide(s), and with a second probe that is complementary to a nucleotide sequence contiguous with the diagnostic portion (the contiguous probe), under conditions, wherein the diagnostic probe remains bound substantially only to the target nucleic acid. The diagnostic and contiguous probes can be of different lengths and/or have different melting temperatures such that the stringency of the hybridization can be adjusted to permit their selective hybridization to the target, wherein the probe having the higher melting temperature is hybridized at higher stringency and, following washing to remove unbound and/or non-selectively bound probe, the other probe having the lower melting temperature is hybridized at lower stringency. The diagnostic probe and contiguous probe are then covalently ligated such as, for example, using T4 DNA ligase, to thereby produce a larger target probe that is complementary to the target sequence, and the probes that are not ligated are removed by modifying the hybridization stringency. In this respect, probes that have not been ligated will selectively hybridize under lower stringency hybridization conditions than probes that have been ligated. Accordingly, the stringency of the hybridization can be increased to a stringency that is at least as high as the stringency used to hybridize the longer probe, and preferably at a higher stringency due to the increased length contributed by the shorter probe following ligation.

In another example, one or both of the probes is labelled such that the presence or absence of the target sequence can be tested by melting the target-probe duplex, eluting the dissociated probe, and testing for the label(s). Where both probes are labelled, different ligands are used to permit distinction between the ligated and unligated probes, in which case the presence of both labels in the same eluate fraction confirms the ligation event. If the target nucleic acid is bound to a solid matrix e.g., in a Southern hybridization, slot blot, dot blot, or microchip assay format, the presence of both the diagnostic and contiguous probes can be determined directly.

Methylation specific microarrays (MSO) may also be used. A suitable method is described, for example, in Adorjan et al. Nucl. Acids Res., 30: e21, 2002. MSO uses nucleic acid that has been contacted with a compound that selectively mutates a non-methylated cytosine residue (e.g., bisulfite) as template for an amplification reaction that amplifies both mutant and non-mutated nucleic acid. The amplification is performed with at least one primer that comprises a detectable label, such as, for example, a fluorophore, e.g., Cy3 or Cy5.

To produce a microarray for detection of mutated nucleic acid oligonucleotides are spotted onto, for example, a glass slide, preferably, with a degree of redundancy (for example, as described in Golub et al., Science, 286:531-537, 1999). Preferably, for each CpG dinucleotide analyzed two different oligonucleotides are used. Each oligonucleotide comprises a sequence N₂-16CGN₂-16 or N₂-16TGN₂-16 (wherein N is a number of nucleotides adjacent or juxtaposed to the CpG dinucleotide of interest) reflecting the methylated or non-methylated status of the CpG dinucleotides.

The labelled amplification products are then hybridized to the oligonucleotides on the microarray under conditions that enable detection of single nucleotide differences. Following washing to remove unbound amplification product, hybridization is detected using, for example, a microarray scanner. Not only does this method allow for determination of the methylation status of a large number of CpG dinucleotides, it is also semi-quantitative, enabling determination of the degree of methylation at each CpG dinucleotide analyzed. As there may be some degree of heterogeneity of methylation in a single sample, such quantification may assist in the diagnosis of cancer.

### Amplification-Based Assay Format

In an alternative example, the hybridization is detected using an amplification system. In methylation-specific PCR formats (MSP; Herman et al. Proc. Natl. Acad. Sci. USA 93:9821-9826, 1992), the hybridization is detected using a process comprising amplifying the bisulfite-treated DNA. Accordingly, by using one or more probe or primer that anneals specifically to the unmutated sequence under moderate and/or high stringency conditions an amplification product is only produced using a sample comprising a methylated nucleotide. Alternate assays that provide for selective amplification of either the methylated or the unmethylated component from a mixture of bisulfite-treated DNA are provided by Cottrell et al., Nucl. Acids Res. 32: e10, 2003 (HeavyMethyl PCR), Rand et al. Nucl. Acids Res. 33:e127, 2005 (Headloop PCR), Rand et al. Epigenetics 1:94-100, 2006 (Bisulfite Differential Denaturation PCR) and PCT/AU07/000389 (End-specific PCR).

Any amplification assay format described herein can be used, such as, for example, polymerase chain reaction (PCR), rolling circle amplification (RCA), inverse polymerase chain reaction (iPCR), in situ PCR (Singer-Sam et al., 1990, supra), strand displacement amplification, or cycling probe technology. PCR techniques have been developed for detection of gene mutations (Kuppuswamy et al., Proc. Natl. Acad. Sci. USA 88:1143-1147, 1991) and quantitation of allelic-specific expression (Szabo and Mann, Genes Dev. 9: 3097-3108, 1995; and Singer-Sam et al., PCR Methods Appl. 1: 160-163, 1992). Such techniques use internal primers, which anneal to a PCR-generated template and terminate immediately 5′ of the single nucleotide to be assayed. Such as format is readily combined with ligase chain reaction as described herein above. The use of a real-time quantitative assay format is also useful. Subject to the selection of appropriate primers, such assay formats are generally described herein above and apply mutatis mutandis to the presently described selective mutagenesis approach.

Methylation-specific melting-curve analysis (essentially as described in Worm et al., Clin. Chem., 47:1183-1189, 2001) is also contemplated by the present invention. This process exploits the difference in melting temperature in amplification products produced using bisulfite treated methylated or unmethylated nucleic acid. In essence, non-discriminatory amplification of a bisulfite treated sample is performed in the presence of a fluorescent dye that specifically binds to double stranded DNA (e.g., SYBR Green I). By increasing the temperature of the amplification product while monitoring fluorescence the melting properties and thus the sequence of the amplification product is determined. A decrease in the fluorescence reflects melting of at least a domain in the amplification product. The temperature at which the fluorescence decreases is indicative of the nucleotide sequence of the amplified nucleic acid, thereby permitting the nucleotide at the site of one or more CpG dinucleotides to be determined.

Aspects of the present invention also encompass the use of real-time quantitative forms of PCR, such as, for example, TaqMan (Holland et al., Proc. Natl. Acad. Sci. USA, 88:7276-7280, 1991; Lee et al., Nucleic Acid Res. 21:3761-3766, 1993) to perform this embodiment. For example, the MethylLight method of Eads et al., Nucl. Acids Res. 28: E32, 2000 uses a modified TaqMan assay to detect methylation of a CpG dinucleotide. Essentially, this method comprises treating a nucleic acid sample with bisulfite and amplifying nucleic acid comprising one or more CpG dinucleotides that are methylated in a neoplastic cell and not in a control sample using an amplification reaction, e.g., PCR. The amplification reaction is performed in the presence of three oligonucleotides, a forward and reverse primer that flank the region of interest and a probe that hybridizes between the two primers to the site of the one or more methylated CpG dinucleotides. The probe is dual labelled with a 5′ fluorescent reporter and a 3′ quencher (or vice versa). When the probe is intact, the quencher dye absorbs the fluorescence of the reporter due to their proximity. Following annealing of to the PCR product the probe is cleaved by 5′ to 3′ exonuclease activity of, for example, Taq DNA polymerase. This cleavage releases the reporter from the quencher thereby resulting in an increased fluorescence signal that can be used to estimate the initial template methylation level. By using a probe or primer that selectively hybridizes to unmutated nucleic acid (e.g., methylated nucleic acid) the level of methylation is determined, e.g., using a standard curve.

Fluorescent reporters for labeling nucleic acids include but are not limited to fluorescein, 6-fluorescein phosphoramidite (6-FAM), 5-carboxytetramethylrhodamine (5-TAMRA), 6-carboxy-4′,5′-dichloro-2′,7′-dimethoxyfluorescein (6-JOE), Cy3, Cy5, Cy5.5, tetrachlorofluorescein (TET), Yakima Yellow®, hexachlorofluorescein (HEX), Texas Red®, TEX615, rhodamine, ATTO dyes, Alexa Fluor® dyes, or IRDyes®, or any combination thereof. Fluorescence quenchers for labeling nucleic acids include but are not limited to Iowa Black® FQ (3′ IABkFQ), Iowa Black® RQ, Black Hole Quencher® (BHQ), Dabcyl, Qxl, or ZEN, or any combination thereof.

Alternatively, rather than using a labelled probe that requires cleavage, a probe, such as, for example, a Molecular Beacon is used (see, for example, Mhlanga and Malmberg, Methods 25:463-471, 2001). Molecular beacons are single stranded nucleic acid molecules with a stem-and-loop structure. The loop structure is complementary to the region surrounding the one or more CpG dinucleotides that are methylated in a neoplastic sample and not in a control sample. The stem structure is formed by annealing two “arms” complementary to each other, which are on either side of the probe (loop). A fluorescent moiety is bound to one arm and a quenching moiety that suppresses any detectable fluorescence when the molecular beacon is not bound to a target sequence is bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base is determined by the level of fluorescence detected. Such an assay facilitates detection of one or more unmutated sites (i.e. methylated nucleotides) in a nucleic acid.

Fluorescently labelled locked nucleic acid (LNA) molecules or fluorescently labelled protein-nucleic acid (PNA) molecules are useful for the detection of nucleotide differences (e.g., as described in Simeonov and Nikiforov, Nucleic Acids Research, 30(17):1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a mutated cytosine in a methylated CpG dinucleotide. Preferably, fluorescently labelled LNA or PNA technology is used to detect at least a single base change in a nucleic acid that has been previously amplified using, for example, an amplification method known in the art and/or described herein.

As will be apparent to the skilled artisan, LNA or PNA detection technology is amenable to a high-throughput detection of one or more markers by immobilizing an LNA or PNA probe to a solid support, as described in Orum et al., Clin. Chem. 45:1898-1905, 1999.

Alternatively, a real-time assay, such as, for example, the so-called HeavyMethyl assay (Cottrell et al., 2003, supra) is used to determine the presence or level of methylation of nucleic acid in a test sample. Essentially, this method uses one or more non-extendible nucleic acid (e.g., oligonucleotide) blockers that bind to bisulfite-treated nucleic acid in a methylation specific manner (e.g., the blocker/s bind specifically to unmutated DNA under moderate to high stringency conditions). An amplification reaction is performed using one or more primers that may optionally be methylation specific but that flank the one or more blockers. In the presence of unmethylated nucleic acid (e.g., non-mutated DNA) the blocker/s bind and no PCR product is produced. Using a TaqMan assay essentially as described supra the level of methylation of nucleic acid in a sample is determined.

Other amplification based methods for detecting methylated nucleic acid following treatment with a compound that selectively mutates a non-methylated cytosine residue include, for example, methylation-specific single stranded conformation analysis (MS-SSCA) (Bianco et al., Hum. Mutat., 14:289-293, 1999), methylation-specific denaturing gradient gel electrophoresis (MS-DGGE) (Abrams and Stanton, Methods Enzymol., 212:71-74, 1992) and methylation-specific denaturing high-performance liquid chromatography (MS-DHPLC) (Deng et al. Chin. J. Cancer Res., 12:171-191, 2000). Each of these methods use different techniques for detecting nucleic acid differences in an amplification product based on differences in nucleotide sequence and/or secondary structure. Such methods are clearly contemplated herein.

As with other amplification-based assay formats, the amplification product is analyzed using a range of procedures, including gel electrophoresis, gel filtration, mass spectrometry, and in the case of labelled primers, by identifying the label in the amplification product. In an alternative embodiment, restriction enzyme digestion of PCR products amplified from bisulfite-converted DNA is performed essentially as described by Sadri and Hornsby, Nucl. Acids Res. 24:5058-5059, 1996; and Xiong and Laird, Nucl. Acids Res. 25:2532-2534, 1997), to analyze the product formed.

High throughput detection methods, such as, for example, matrix-assisted laser desorption/ionization time of flight (MALDI-TOF), electrospray ionization (ESI), Mass spectrometry (including tandem mass spectrometry, e.g. LC MS/MS), biosensor technology, evanescent fiber-optics technology or DNA chip technology, can also be employed.

As with the other assay formats described herein that utilize hybridization and/or amplification detection systems, combinations of such processes as described herein above are particularly contemplated by the selective mutagenesis-based assay formats of the present invention. In one example, the increased methylation is detected by performing a process comprising:
(i) contacting the nucleic acid with an amount of a compound that selectively mutates a non-methylated cytosine residue within a CpG dinucleotide under conditions sufficient to induce mutagenesis thereby producing a mutated nucleic acid;
(ii) hybridizing the nucleic acid to two non-overlapping and non-complementary primers each of which comprises a nucleotide sequence that is complementary to a sequence in the DNA comprising a methylated cytosine residue under conditions such that hybridization to the non-mutated nucleic acid occurs;
(iii) amplifying nucleic acid intervening the hybridized primers thereby producing a DNA fragment consisting of a sequence that comprises a primer sequence;
(iv) hybridizing the amplified DNA fragment to a probe comprising a nucleotide sequence that corresponds or is complementary to a sequence comprising a methylated cytosine residue under conditions such that hybridization to the non-mutated nucleic acid occurs; and detecting the hybridization.

### Negative Read-Out Assays

In another example, the assay format comprises a negative read-out system in which reduced methylation of DNA from a healthy/normal control sample is detected as a positive signal and preferably, methylated DNA from a neoplastic sample is not detected or is only weakly detected.

In a preferred embodiment, the reduced methylation is determined using a process comprising:
(i) contacting the nucleic acid with an amount of a compound that selectively mutates a non-methylated cytosine residue within a CpG island under conditions sufficient to induce mutagenesis thereby producing a mutated nucleic acid;
(ii) hybridizing the nucleic acid to a probe or primer comprising a nucleotide sequence that is complementary to a sequence comprising the mutated cytosine residue under conditions such that selective hybridization to the mutated nucleic acid occurs; and
(iii) detecting the selective hybridization.

In this context, the term “selective hybridization” means that hybridization of a probe or primer to the mutated nucleic acid occurs at a higher frequency or rate, or has a higher maximum reaction velocity, than hybridization of the same probe or primer to the corresponding non-mutated sequence. Preferably, the probe or primer does not hybridize to the methylated sequence (or non-mutated sequence) under the reaction conditions used.

### Hybridization-Based Assay Format

In one embodiment the hybridization is detected using Southern, dot blot, slot blot or other nucleic acid hybridization means (Kawai et al., 1994, supra; Gonzalgo et al., 1997, supra). Subject to appropriate probe selection, such assay formats are generally described herein above and apply mutatis mutandis to the presently described selective mutagenesis approach. Preferably, a ligase chain reaction format is employed to distinguish between a non-mutated and mutated nucleic acid. In this respect, the assay requirements and conditions are as described herein above for positive read-out assays and apply mutatis mutandis to the present format. However, the selection of probes will differ. For negative read-out assays, one or more probes are selected that selectively hybridize to the mutated sequence rather than the non-mutated sequence.

Preferably, the ligase chain reaction probe(s) have 3-terminal and/or 5′-terminal sequences that comprise a CpG dinucleotide that is not methylated in a healthy control sample, but is hypermethylated in cancer, such that the diagnostic probe and contiguous probe are capable of being ligated only when the cytosine of the CpG dinucleotide is mutated to thymidine e.g., in the case of a non-methylated cytosine residue.

As will be apparent to the skilled artisan the MSO method described supra is amenable to either or both positive and/or negative readout assays. This is because the assay described detects both mutated and non-mutated sequences thereby facilitating determining the level of methylation. However, an assay detecting only methylated or non-methylated sequences is contemplated herein.

### Amplification-Based Assay Format

In an alternative example, the hybridization is detected using an amplification system using any amplification assay format as described herein above for positive read-out assay albeit using primers (and probes where applicable) selectively hybridize to a mutated nucleic acid.

In adapting the HeavyMethyl assay described supra to a negative read-out format, the blockers that bind to bisulfite-treated nucleic acid in a methylation specific manner bind specifically to mutated DNA under moderate to high stringency conditions. An amplification reaction is performed using one or more primers that may optionally be methylation specific (e.g., only bind to mutated nucleic acid) but that flank the one or more blockers. In the presence of methylated nucleic acid (e.g., mutated DNA) the blocker/s bind and no PCR product is produced.

In one example, the reduced methylation in the normal/healthy control subject is detected by performing a process comprising:
(i) contacting the nucleic acid with an amount of a compound that selectively mutates non-methylated cytosine residues under conditions sufficient to induce mutagenesis thereby producing a mutated nucleic acid;
(ii) hybridizing the nucleic acid to two non-overlapping and non-complementary primers each of which comprises a nucleotide sequence that is complementary to a sequence in the DNA comprising a mutated cytosine residue under conditions such that hybridization to the mutated nucleic acid occurs;
(iii) amplifying nucleic acid intervening the hybridized primers thereby producing a DNA fragment consisting of a sequence that comprises a primer sequence;
(iv) hybridizing the amplified DNA fragment to a probe comprising a nucleotide sequence that corresponds or is complementary to a sequence comprising a mutated cytosine residue under conditions such that hybridization to the mutated nucleic acid occurs; and
(v) detecting the hybridization.

As will be apparent to the skilled artisan a negative read-out assay preferably includes a suitable control sample to ensure that the negative result is caused by methylated nucleic acid rather than a reaction failing.

This invention also provides kits for the detection and/or quantification of the diagnostic sequences of the invention, or expression or methylation thereof using the methods described herein.

For kits for detection of methylation, the kits of the invention can comprise at least one polynucleotide that hybridizes to at least one of the diagnostic sequences of the invention and at least one reagent for detection of gene methylation. Reagents for detection of methylation include, e.g., sodium bisulfite, polynucleotides designed to hybridize to sequence that is the product of a biomarker sequence of the invention if the biomarker sequence is not methylated (e.g., containing at least one C→U conversion), and/or a methylation-sensitive or methylation-dependent restriction enzyme. The kits may also include control natural or synthetic DNA sequences representing methylated or unmethylated forms of the sequence. The kits can provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

As detailed hereinbefore, hypermethylation is associated with transcriptional silencing. Accordingly, in addition to the increased level of methylation of these genes providing a basis upon which to screen for the predisposition to or onset of an esophageal or gastric neoplasm, the downregulation in the level of expression of these genes is also diagnostically valuable. In accordance with this aspect of the present invention, reference to a gene “expression product” or “expression of a gene” is a reference to either a transcription product (such as primary RNA or mRNA) or a translation product such as protein. In this regard, one can assess changes to the level of expression of a gene either by screening for changes to the level of expression product which is produced (e.g., RNA or protein), changes to the chromatin proteins with which the gene is associated, for example the presence of histone H3 methylated on lysine at amino acid position number 9 or 27 (repressive modifications) or changes to the DNA itself which acts to downregulate expression, such as changes to the methylation of the DNA. These genes and their gene expression products, whether they be RNA transcripts, changes to the DNA which act to downregulate expression or encoded proteins, are collectively referred to as “neoplastic markers”.

Accordingly, another aspect of the present invention is directed to a method of screening for the onset or predisposition to the onset of or monitoring an esophagus or gastric neoplasm in an individual, said method comprising assessing the level of expression of a DNA region selected from:
(i) the region, including 2 kb upstream of the transcription start site, defined by Hg19 coordinates:
(1) chr12:24962958 . . . 25102393; and/or
(2) chr7:50344378 . . . 50472798; o
(ii) the gene region, including 2 kb upstream of:
(1) BCAT1; and/or (2) IKZF1
in a biological sample from said individual wherein a lower level of expression of at least one of the DNA regions of group (i) and/or (ii) relative to control levels is indicative of an esophagus or stomach neoplasm or a predisposition to the onset of a neoplastic state.

In one embodiment said control level is a non-neoplastic level.

In another embodiment, said method is directed to screening for either BCAT1 or IKZF1 expression in a biological sample.

In still another embodiment, said method is directed screening for both BCAT1 and IKZF1 expression in a biological sample.

In yet another embodiment, said method is directed to screening for both BCAT1 and IKZF1 expression but wherein only one of said genes exhibits a lower level of expression.

In a further embodiment, said method is directed to screening for both BCAT1 and IKZF1 expression but wherein both of said genes exhibits a lower level of expression.

In still another embodiment, the neoplasm is malignant, such as an adenocarcinoma.

In a further embodiment, the neoplasm is non-malignant, such as an adenoma or dysplasia.

The method of this aspect of the present invention is predicated on the comparison of the level of the neoplastic markers of a biological sample with the control levels of these markers. The “control level” may be either a “normal level”, which is the level of marker expressed by a corresponding esophageal or gastric cell or cellular population which is not neoplastic or it may be the level of marker expression of a previously analyzed sample, such as would occur in the context of monitoring.

As detailed hereinbefore, the normal (or “non-neoplastic”) level may be determined using tissues derived from the same individual who is the subject of testing. However, it would be appreciated that this may be quite invasive for the individual concerned and it is therefore likely to be more convenient to analyse the test results relative to a standard result which reflects individual or collective results obtained from individuals other than the patient in issue.

As detailed hereinbefore, the present invention is designed to screen for a neoplastic cell or cellular population, which is located in the esophagus or stomach. Accordingly, reference to “cell or cellular population” should be understood as a reference to an individual cell or a group of cells. Said group of cells may be a diffuse population of cells, a cell suspension, an encapsulated population of cells or a population of cells which take the form of tissue.

Reference to “expression” should be understood as a reference to the transcription and/or translation of a nucleic acid molecule. Reference to “RNA” should be understood to encompass reference to any form of RNA, such as primary RNA or mRNA or non-translated RNA (e.g. miRNAs etc.). Without limiting the present invention in any way, the modulation of gene transcription leading to increased or decreased RNA synthesis may also correlate with the translation of some of these RNA transcripts (such as mRNA) to produce a protein product. Accordingly, the present invention also extends to detection methodology which is directed to screening for modulated levels or patterns of the neoplastic marker protein products as an indicator of the neoplastic state of a cell or cellular population. Although one method is to screen for mRNA transcripts and/or the corresponding protein product, it should be understood that the present invention is not limited in this regard and extends to screening for any other form of neoplastic marker expression product such as, for example, a primary RNA transcript.

Reference to “nucleic acid molecule” should be understood as a reference to both deoxyribonucleic acid molecules and ribonucleic acid molecules and fragments thereof. The present invention therefore extends to both directly screening for mRNA levels in a biological sample or screening for the complementary cDNA which has been reverse-transcribed from an mRNA population of interest. It is well within the skill of the person of skill in the art to design methodology directed to screening for either DNA or RNA. As detailed above, the method of the present invention also extends to screening for the protein product translated from the subject mRNA or the genomic DNA itself.

In one preferred embodiment, the level of gene expression is measured by reference to genes which encode a protein product and, more particularly, said level of expression is measured at the protein level.

The present invention should be understood to encompass methods of detection based on identifying both proteins and/or nucleic acid molecules in one or more biological samples. This may be of particular significance to the extent that some of the neoplastic markers of interest may correspond to genes or gene fragments which do not encode a protein product. Accordingly, to the extent that this occurs it would not be possible to test for a protein and the subject marker would have to be assessed on the basis of transcription expression profiles or changes to genomic DNA.

The term “protein” should be understood to encompass peptides, polypeptides and proteins (including protein fragments). The protein may be glycosylated or unglycosylated and/or may contain a range of other molecules fused, linked, bound or otherwise associated to the protein such as amino acids, lipids, carbohydrates or other peptides, polypeptides or proteins. Reference herein to a “protein” includes a protein comprising a sequence of amino acids as well as a protein associated with other molecules such as amino acids, lipids, carbohydrates or other peptides, polypeptides or proteins.

The proteins encoded by the neoplastic markers of the present invention may be in multimeric form meaning that two or more molecules are associated together. Where the same protein molecules are associated together, the complex is a homomultimer. An example of a homomultimer is a homodimer. Where at least one marker protein is associated with at least one non-marker protein, then the complex is a heteromultimer such as a heterodimer.

Means of assessing the subject expressed neoplasm markers in a biological sample can be achieved by any suitable method, which would be well known to the person of skill in the art. To this end, it would be appreciated that to the extent that one is examining either a homogeneous cellular population (such as a tumour biopsy or a cellular population which has been enriched from a heterogeneous starting population) or a tissue section, one may utilise a wide range of techniques such as in situ hybridisation, assessment of expression profiles by microassays, immunoassays and the like (hereinafter described in more detail) to detect the absence of or downregulation of the level of expression of one or more markers of interest. However, to the extent that one is screening a heterogenous cellular population or a bodily fluid in which heterogeneous populations of cells are found, such as a blood sample, the absence of or reduction in level of expression of a particular marker may be undetectable due to the inherent expression of the marker by non-neoplastic cells which are present in the sample. That is, a decrease in the level of expression of a subgroup of cells may not be detectable. In this situation, a more appropriate mechanism of detecting a reduction in a neoplastic subpopulation of the expression levels of one or more markers of the present invention is via indirect means, such as the detection of epigenetic changes.

Methods of detecting changes to gene expression levels (in addition to the methylation analyses hereinbefore described in detail), particularly where the subject biological sample is not contaminated with high numbers of non-neoplastic cells, include but are not limited to:
(i) In vivo detection.

Molecular Imaging may be used following administration of imaging probes or reagents capable of disclosing altered expression of the markers in the intestinal tissues.

Molecular imaging (Moore et al., BBA, 1402:239-249, 1988; Weissleder et al., Nature Medicine 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy.
(ii) Detection of downregulation of RNA expression in the cells by Fluorescent In Situ

Hybridization (FISH), or in extracts from the cells by technologies such as Quantitative Reverse Transcriptase Polymerase Chain Reaction (QRTPCR) or Flow cytometric qualification of competitive RT-PCR products (Wedemeyer et al., Clinical Chemistry 48:9 1398-1405, 2002).
(iii) Assessment of expression profiles of RNA, for example by array technologies (Alon et al., Proc. Natl. Acad. Sci. USA: 96:6745-6750, June 1999).

A “microarray” is a linear or multi-dimensional array of preferably discrete regions, each having a defined area, formed on the surface of a solid support. The density of the discrete regions on a microarray is determined by the total numbers of target polynucleotides to be detected on the surface of a single solid phase support. As used herein, a DNA microarray is an array of oligonucleotide probes placed onto a chip or other surfaces used to amplify or clone target polynucleotides. Since the position of each particular group of probes in the array is known, the identities of the target polynucleotides can be determined based on their binding to a particular position in the microarray.

DNA microarray technology make it possible to conduct a large scale assay of a plurality of target nucleic acid molecules on a single solid phase support. U.S. Pat. No. 5,837,832 (Chee et al.) and related patent applications describe immobilizing an array of oligonucleotide probes for hybridization and detection of specific nucleic acid sequences in a sample. Target polynucleotides of interest isolated from a tissue of interest are hybridized to the DNA chip and the specific sequences detected based on the target polynucleotides' preference and degree of hybridization at discrete probe locations. One important use of arrays is in the analysis of differential gene expression, where the profile of expression of genes in different cells or tissues, often a tissue of interest and a control tissue, is compared and any differences in gene expression among the respective tissues are identified. Such information is useful for the identification of the types of genes expressed in a particular tissue type and diagnosis of conditions based on the expression profile.
(iv) Measurement of altered neoplastic marker protein levels in cell extracts, for example by immunoassay.

Testing for proteinaceous neoplastic marker expression product in a biological sample can be performed by any one of a number of suitable methods which are well known to those skilled in the art. Examples of suitable methods include, but are not limited to, antibody screening of tissue sections, biopsy specimens or bodily fluid samples. To the extent that antibody based methods of diagnosis are used, the presence of the marker protein may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.
(v) Determining altered expression of protein neoplastic markers on the cell surface, for example by immunohistochemistry.
(vi) Determining altered protein expression based on any suitable functional test, enzymatic test or immunological test in addition to those detailed in points (iv) and (v) above.

A person of ordinary skill in the art could determine, as a matter of routine procedure, the appropriateness of applying a given method to a particular type of biological sample.

A related aspect of the present invention provides a molecular array, which array comprises a plurality of:
(i) nucleic acid molecules comprising a nucleotide sequence corresponding to any two or more of the neoplastic marker DNA hereinbefore described or a sequence exhibiting at least 80% identity thereto or a functional derivative, fragment, variant or homologue of said nucleic acid molecule; or
(ii) nucleic acid molecules comprising a nucleotide sequence capable of hybridising to any one or more of the sequences of (i) under medium stringency conditions or a functional derivative, fragment, variant or homologue of said nucleic acid molecules; or
(iii) nucleic acid probes or oligonucleotides comprising a nucleotide sequence capable of hybridising to any two or more of the sequences of (i) under medium stringency conditions or a functional derivative, fragment, variant or homologue of said nucleic acid molecule; or
(iv) probes capable of binding to any two or more of the proteins encoded by the nucleic acid molecules of (i) or a derivative, fragment or, homologue thereof
wherein the level of expression of said marker genes of (i)-(iii) or proteins of (iv) is indicative of the neoplastic state of a cell or cellular subpopulation derived from the esophagus or stomach.

Preferably, said percent identity is at least 85%, 90%, 91%, 92%, 93%, 94%, 95%, 96%, 97%, 98% or 99%.

“Hybridization” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

Reference herein to a low stringency includes and encompasses from at least 0% or at least about 0% to at least 15% or at least about 15% v/v formamide and from at least 1 M or at least about 1 M to at least 2 M or at least about 2 M salt for hybridization, and at least 1 M or at least about 1 M to at least 2M or at least about 2 M salt for washing conditions. Generally, low stringency is at from 25-30° C. or about 25-30° C. to 42° C. or about 42° C. The temperature may be altered and higher temperatures used to replace formamide and/or to give alternative stringency conditions. Alternative stringency conditions may be applied where necessary, such as medium stringency, which includes and encompasses from at least 16% v/v or at least about 16% v/v to at least 30% v/v or at least about 30% v/v formamide and from at least 0.5 M or at least about 0.5 M to at least 0.9 M or at least about 0.9 M salt for hybridization, and at least 0.5 M or at least about 0.5 M to at least 0.9 M or at least about 0.9 M salt for washing conditions, or high stringency, which includes and encompasses from at least 31% v/v or at least about 31% v/v to at least 50% v/v or at least about 50% v/v formamide and from at least 0.01 M or at least about 0.01 M to at least 0.15 M or at least about 0.15 M salt for hybridization, and at least 0.01 M or at least about 0.01 M to at least 0.15 M or at least about 0.15 M salt for washing conditions. In general, washing is carried out Tₘ=69.3+0.41 (G+C)% (Marmur and Doty, J. Mol. Biol. 5:109, 1962). However, the Tₘ of a duplex DNA decreases by 1° C. with every increase of 1% in the number of mismatch base pairs (Bonner and Laskey, Eur. J. Biochem. 46:83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Where nucleic acids of the invention are defined by their ability to hybridize under high stringency, these conditions comprise 50% or about 50% formamide at 37° C. or about 37° C. to 42° C. or about 42° C. In one aspect, nucleic acids of the invention are defined by their ability to hybridize under reduced stringency comprising conditions in 35% or about 35% to 25% or about 25% formamide at 30° C. or about 30° C. to 35° C. or about 35° C. Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Preferably, the subject probes are designed to bind to the nucleic acid or protein to which they are directed with a level of specificity which minimises the incidence of non-specific reactivity. However, it would be appreciated that it may not be possible to eliminate all potential cross-reactivity or non-specific reactivity, this being an inherent limitation of any probe based system.

In terms of the probes which are used to detect the subject proteins, they may take any suitable form including antibodies and aptamers.

A library or array of nucleic acid or protein probes provides rich and highly valuable information. Further, two or more arrays or profiles (information obtained from use of an array) of such sequences are useful tools for comparing a test set of results with a reference, such as another sample or stored calibrator. In using an array, individual probes typically are immobilized at separate locations and allowed to react for binding reactions. Primers associated with assembled sets of markers are useful for either preparing libraries of sequences or directly detecting markers from other biological samples.

A library (or array, when referring to physically separated nucleic acids corresponding to at least some sequences in a library) of gene markers exhibits highly desirable properties. These properties are associated with specific conditions, and may be characterized as regulatory profiles. A profile, as termed here refers to a set of members that provides diagnostic information of the tissue from which the markers were originally derived. A profile in many instances comprises a series of spots on an array made from deposited sequences.

A characteristic patient profile is generally prepared by use of an array. An array profile may be compared with one or more other array profiles or other reference profiles. The comparative results can provide rich information pertaining to disease states, developmental state, receptiveness to therapy and other information about the patient.

Another aspect of the present invention provides a diagnostic kit for assaying biological samples comprising one or more agents for detecting one or more neoplastic markers and reagents useful for facilitating the detection by said agents. Further means may also be included, for example, to receive a biological sample. The agent may be any suitable detecting molecule.

The invention is generally disclosed herein using affirmative language to describe the numerous embodiments. The invention also includes embodiments in which subject matter is excluded, in full or in part, such as substances or materials, method steps and conditions, protocols, or procedures.

The present invention is further described by reference to the following non-limiting examples. Those in the art will appreciate that many other embodiments also fall within the scope of the invention, as it is described herein above and in the claims.

### EXAMPLES

### Example 1: Analysis of Esophageal Cancers

### Study Cohort

An observational study was performed which collected blood from patients with invasive adenocarcinoma of the prostate (n=94), breast (32), esophagus (49) or colon/rectum (211), prior to any treatment or resection of primary cancers, and from 253 clinically assessed controls with no known prior or current adenocarcinoma, Table 1. In addition, biopsies were collected from cancer and adjacent non-neoplastic tissues either prior to treatment or at surgery from patients with prostate (n=9), breast (26), esophageal (6) and colon/rectum (15) cancer.

### Methods

DNA was extracted from 4 mL K3EDTA plasma and 10-20 mg tissue and bisulphite converted. All DNA samples were assayed for methylated BCAT1 and IKZF1 DNA using a real-time multiplex PCR assay. A total of 5 ng bisulphite converted tissue DNA was assayed for methylation in BCAT1 and IKZF1 and the level of expression was expressed as the percentage of the total input (determined with ACTB). Tissue samples were considered positive if the % methylation of either gene was above the 75^{th} percentile of non-neoplastic tissue levels. Blood samples were considered positive with any detectable signal of either methylation marker. Real-time PCR was performed according to the protocol described in Table 5. Probes and primers used for BCAT1 and IKZF1 PCR are described in Table 6.

### Results

### Tissue

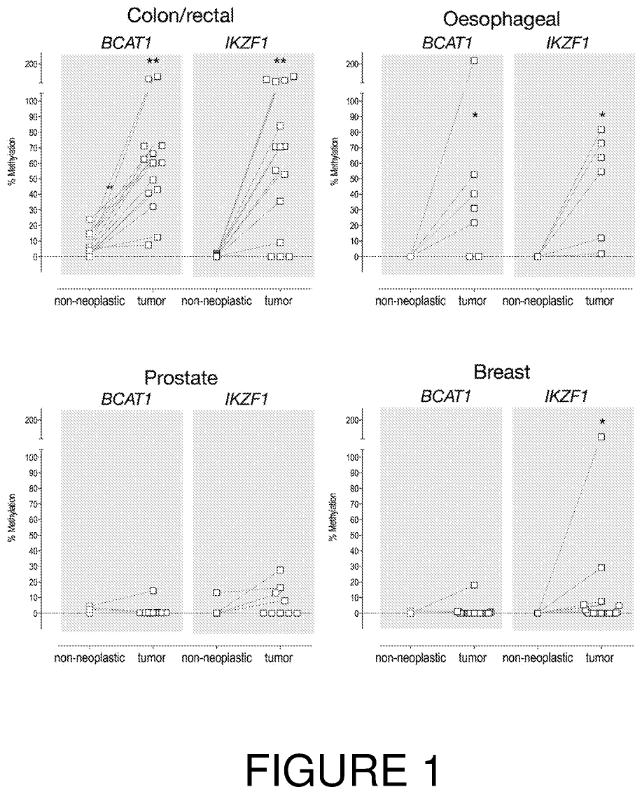

Colorectal cancer (CRC) and esophageal cancer tissues had significantly higher methylation levels compared to matched non-neoplastic tissue for both BCAT1 and IKZF1, FIG. 1 (top panel)

The median (or interquartile range, IQR) methylation levels in prostate (BCAT1, 0.3%; IKZF1, 0.1%) and breast (0%) cancer tissues were 2-3 orders of magnitude less than the levels measured in CRC (60.4%; 70.7%) and esophageal (31.0; 63.7%) cancer tissues, Table 1 & FIG. 1 (bottom panel).

Using the 75^{th} percentiles measured in non-neoplastic tissues as positivity cutoffs, the cancer tissue positivity rates were: CRC, 14/15, 93.3% (95% CI: 68.1-99.8); esophageal, 6/6, 100% (54.1-100); prostate, 4/9, 44.4% (13.7-78.8); breast, 11/26, 42.3% (23.4-63.1).

### Blood

Circulating tumor DNA (ctDNA) positivity rates were significantly higher in CRC (126/211, 59.7%, 95% CI: 52.8-66.4) and esophageal cancer (26/49, 53.1%, 38.3-67.5) cases only compared to controls (16/253, 6.3%, 3.7-10.1; p<0.01).

ctDNA was more likely to be positive in late stage cancers (Table 2). There were no significant associations of the other cancers and positivity status with any patient demographics or tumor features (p>0.05).

### Conclusion

Only colorectal and esophageal cancer patients had significantly higher ctDNA positivity rates (using methylated BCAT1/IKZF1) compared to controls. This was also reflected in a higher proportion of cases showing methylation in the cancer tissue.

The methylated BCAT1/IKZF1 blood test should be investigated further as a screening and surveillance tool for esophageal cancer, as well as its existing use for CRC.

TABLE 1
Study Cohort

| | | Age (median, IQR), |
|---|---|---|
| Group | No. Male (%) | years |
| Control | 92/253 (36.4%) | 57.7 (51.3-65.2) |
| Colorectal Cancer | 132/211 (62.6%) | 68.6 (60.6-76.4) |
| Oesophageal Cancer | 40/49 (82%) | 69.8 (59.6-75.3) |
| Prostate Cancer | 94/94 (100%) | 67.5 (61.1-72.9) |
| Breast Cancer | 0/32 (0%) | 63.7 (52.5-70.1) |

TABLE 2
Blood Positivity in Early v Late Stage Cancers

| | | | | No. of |
|---|---|---|---|---|
| | No. of positive/total (%) | | | positive/total |
| Group | Early Stage | Late Stage | P-value | (%) |
| Control | N/A | N/A | N/A | 16/253 (6.3) |
| Colorectal | 44/107 (41.1) | 82/104 (78.8 | <0.0001 | 126/211 (59.7) |
| Cancer | | | | |
| Oesophageal | 5/18 (27.8) | 21/31 (67.7) | 0.007 | 26/49 (53.1) |
| Cancer | | | | |
| Prostate Cancer* | 6/87 (6.9) | 1/6 (16.7) | 0.380 | 7/94 (7.4) |
| Breast Cancer | 3/29 (10.3) | 0/3 (0) | 0.559 | 3/32 (9.4) |

*1 prostate cancer was unstaged

### Example 2: Analysis of Gastric Cancers

### Methylation of Bcat1 and Ikzf1 in Cancer and Non-Cancer Tissue Specimens

All cancer tissues had significantly higher methylation levels than the adjacent non-tumour tissue (Chi² test, p<0.05). Cancer tissue positivity rates in gastric cancer tissue were high (9/12, 75.0%) (Table 3).

TABLE 3

| | Normal tissue | Cancer tissue |
|---|---|---|
| Gastric | 1/12 (8.3%) | 9/12 (75%) |
| Adenocarcinoma | | |

### Methylation of Bcat1 and Ikzf1 in Blood Samples

High positivity rates of blood samples for the methylated biomarkers were observed in gastric cancer patients (Table 4).

TABLE 4
Blood Positivity for Methylated BCAT1
and IKZF1 in Adenocarcinomas

| | Positivity rate, |
|---|---|
| | Number of Positive/Total |
| Cancer Type | (%) |
| Healthy Control | 16/253 (6.3%) |
| Gastric | 9/22 (40.9%)* |

*p < 0.05 compared to control

### Example 3: Detailed Methods for Examples 1 and 2

### Methods

Venous blood (18 ml) was collected into K₃EDTA Vacuette tubes (Greiner Bio-One, Frickenhausen, Germany) from participants. Blood tubes were kept at 4° C. prior to plasma processing (not 44 h from blood collection). Plasma was prepared by centrifugation at 1,500 g for 10 min at 4° C. (deceleration at lowest setting), followed by retrieval of the plasma fraction and a repeat centrifugation. The resulting plasma was stored at −80° C. Frozen plasma samples were shipped on dry ice to Clinical Genomics Technologies (Sydney, Australia) and stored at −80° C. until testing.

Blood DNA methylation testing. All plasma samples of at least 3.9 ml were assayed at Clinical Genomics Technologies for the presence of methylated BCAT1 and IKZF1 DNA. Samples were processed and assayed in batches of 22 samples plus two process controls as described in Pedersen S, Symonds E, Baker R et al. (Evaluation of an assay for methylated BCAT1 and IKZF1 in plasma for detection of colorectal neoplasia. BMC Cancer 2015; 15: 654) but with the following changes: the bisulphite conversion setup and subsequent purification was automated on a QIAcube HT instrument (Qiagen, Hilden, Germany) and the IKZF1 component in the methylation-specific PCR assay was modified to enable detection of partially methylated IKZF1 target regions (Table 5). Bisulfite-converted DNA from each plasma sample was assayed in triplicate with real-time PCR performed on a Light Cycler 480 II instrument (Roche Diagnostics, IN, USA). A sample was deemed qualitatively positive if at least one PCR replicate was positive for either BCAT1 or IKZF1 DNA methylation.

Statistical analyses. The main outcome measure was positivity rate by diagnosis. Binomial distribution was assumed for calculations of 95% confidence interval (95% CI). Differences in paired positivity proportions and concordance analyses were analyzed using McNemar's test, whereas differences in non-paired proportions used a χ²-test (two-tailed; significant level, 0.05). Potential confounding co-variables (age, gender) were analyzed by multiple logistic regression analysis. Test sensitivity estimates were expressed as the ratio of true positives over the sum of true positives plus false negatives. Specificity was estimated as 1-positivity rate in cases with no CRC. As a fecal immunochemical test (FIT) is quantitative and the cutoff for positivity can be varied, test comparison was facilitated by undertaking receiver operating characteristic curve analysis and estimating relative true-positive rates (and hence sensitivity) at an equivalent specificity to the blood DNA test. The GraphPad online scientific software tool was used for the statistical analyses described above. P values<0.05 were considered statistically significant.

### Primers and Probes

The wild-type DNA sequence of the BCAT1 PCR target amplicon is located on Chromosome 12, minus strand; 25,101,992⇒25,102,093 (GRCh37/hg19) gtcttcctgc tgatgcaatc cgctaggtcg cgagtctccg ccgcgagagg gccggtctgc aatccagccc gccacgtgta ctcgccgccg cctcgggcac tg (SEQ ID NO: 1).

The wild-type DNA sequence of the IKZF1 PCR target amplicon is located on Chromosome 7, plus strand; 50,343,867⇒50,343,961 (GRCh37/hg19) gacgacgcac cctctccgtg tcccgctctg cgcccttctg cgcgccccgc tccctgtacc ggagcagcga tccgggaggc ggccgagagg tgcgc (SEQ ID NO: 2)

### PCR Protocols Used to Measure the Methylation Levels Across Ikzf1 and Bcat1 in Specimens

TABLE 5
Real-time PCR protocols BCAT1 and IKZF1

| | BCAT1 | IKZF1 |
|---|---|---|
| FWD | 5′GTTTTTTT | 5′GACGACGTATTTTTTTCGTGT |
| Primer | GTTGATGTAATTCGTTAGGTC | TTC |
| | (SEQ ID NO: 3) | (SEQ ID NO: 4) |
| | 200 nM final | 200 nM final |
| REV | 5′CAATACCCGAAACGACGACG | 5′GCGCACCTCTCGACCG |
| primer | (SEQ ID NO: 5) | (SEQ ID NO: 6) |
| | 200 nM final | 200 nM final |
| Probe | 5′/HEX/ | 5′/FAM/TTTGTATYG/ZEN/GAGT |
| | TTCGTCGCGAGAGGGTCGGTT/ | AGYGATTYGGGAGG/3IABKFQ/ |
| | BHQ/ (SEQ ID NO: 7) | (SEQ ID NO: 8) |
| | 100 nM final | 100 nM final |
| 1× PCR | QuantiTect No Rox Mastermix | QuantiTect No Rox Mastermix |

LC480 cycling conditions

| | | |
|---|---|---|
| 1× | 95° C.; 15 min | 95° C.; 2 min |
| 50× | 95° C.; 15 sec | 95° C.; 15 sec |
| | 62° C.; 40 sec (acquisition) | 62° C.; 40 sec (acquisition) |
| 1× | 40° C.; 10 sec | 40° C.; 10 sec |

TABLE 6

| | | Current sub- | | Resulting bisulphite | Oligonucleotide |
|---|---|---|---|---|---|
| | | region(s) | Coordinates of | converted sequence | sequences for |
| | Genomic | of interest | sub-region(s) | (strands no | measurement of |
| Gene | Strand | (genomic sequence) | (GRCh37/hg19) | longer complementary) | methylation levels |
| BCAT1 | top | 5′cagtgccCGaggCG | 25,101,992- | 5′tagtgttCGaggCGgCGgCG | |
| | strand | gCGgCGagtacaCGt | 25,102,093 | agtataCGtggCGggttggattgta | |
| | | ggCGggctggattgcag | | gatCGgttttttCGCGgCGgag | |
| | | acCGgccctctCGCG | | attCGCGatttagCGgattgtatta | |
| | | gCGgagactCGCGac | | gtaggaagat (SEQ ID NO: | |
| | | ctagCGgattgcatcagc | | 10) | |
| | | aggaagac (SEQ ID | | | |
| | | NO: 9) | | | |
| | minus | 3′gtcacggGCtccGCc | | 3′gttatggGCtttGCtGCtGCtt | 5′gtttttttgttgatgtaattc |
| | strand | GCcGCtcatgtGCacc | | atgtGCattGCttgatttaatgtt | gttaggtc (SEQ ID NO: 3) |
| | | GCccgacctaacgtctg | | tgGCtgggagaGCGCtGCttttga | 5′caatacccgaaacgacgac |
| | | GCcgggagaGCGCc | | GCGCtggattGCttaatgtagttgt | g (SEQ ID NO: 5) |
| | | GCctctgaGCGCtgga | | ttttttg (SEQ ID NO: 12) | 5′ttcgtcgcgagagggtcgg |
| | | tcGCctaacgtagtcgtcc | | | tt (SEQ ID NO: 13) |
| | | ttctg (SEQ ID NO: | | | 5′tttttgttgatgtaattcgtt |
| | | 11) | | | aggtc (SEQ ID NO: 14) |
| | | | | | 5′attacaaaccgaccctctcg |
| | | | | | (SEQ ID NO: 15) |
| | top | 5′agatcccaagggtCGt | 25,101,909- | This sequence is for | 5′agatcccaagggtcgtagc |
| | strand | agcccctggcCGtgtgg | 25,101,995 | measuring CpG | (SEQ ID NO: 17) |
| | | acCGggtctgCGgctg | | methylation levels using | 5′actgccccaggtcttgct |
| | | cagagCGCGgtccCG | | methylation sensitive | (SEQ ID NO: 18) |
| | | gctgcagcaagacctggg | | restriction enzymes (e.g. | |
| | | gcagt (SEQ ID NO: | | HbaII, HhaI (underlined) | |
| | | 16) | | | |
| | minus | 3′tctagggttcccaGCat | | | |
| | strand | cggggaccgGCacacct | | | |
| | | gGCccagacGCcgac | | | |
| | | gtctcGCGCcaggGC | | | |
| | | cgacgtcgttctggacccc | | | |
| | | gtca (SEQ ID NO: | | | |
| | | 19) | | | |
| IKZF1 | top | 5′gaCGaCGcaccctct | 50,343,867- | 5′gaCGaCGtatttttttCGtgttt | 5′gacgacgtatttttttcgtgt |
| | strand | cCGtgtccCGctctgC | 50,343,961 | CGttttgCGtttttttgCGCGtttC | ttc (SEQ ID NO: 4) |
| | | GcccttctgCGCGccc | | GttttttgtatCGgagtagCGattC | 5′gcgcacctctcgaccg |
| | | CGctccctgtacCGgag | | GggaggCGgtCGagaggtgCG | (SEQ ID NO: 5) |
| | | cagCGatcCGggagg | | t (SEQ ID NO: 20) | 5′tttgtatcggagtagcgattc |
| | | CGgcCGagaggtgCG | | | gggag (SEQ ID NO: |
| | | C (SEQ ID NO: 2) | | | 21) |
| | minus | 3′ctGCtGCgtgggaga | | 3′ttGCtGCgtgggagagGCata | |
| | strand | gGCacaggGCgagac | | ggGCgagatGCgggaagatGC | |
| | | GCgggaagacGCGC | | GCgggGCgagggatatgGCttt | |
| | | gggGCgagggacatgG | | gttGCtagGCtttttGCtgGCtttt | |
| | | CctcgtcGCtagGCcct | | tatGCg (SEQ ID NO: 23) | |
| | | ccGCcgGCtctccacG | | | |
| | | CG (SEQ ID NO: | | | |
| | | 22) | | | |
| | top | 5′cCGgagttgCGgctg | 50,343,804- | This sequence is for | 5′ggagttgcggctgagac |
| | strand | agaCGCGCGcCGC | 50,343,895 | measuring CpG | (SEQ ID NO: 25) |
| | | GCGagcCGggggact | | methylation levels using | 5′agagcgggacacggaga |
| | | CGgCGaCGgggCG | | methylation sensitive | (SEQ ID NO: 26) |
| | | gggaCGggaCGaCG | | restriction enzymes (e.g. | |
| | | caccctctcCGtgtccC | | HbaII, Hhal (underlined) | |
| | | Getct (SEQ ID NO: | | | |
| | | 24) | | | |
| | minus | 3′gGCctcaacGCcga | | | |
| | strand | ctctGCGCGCgGCG | | | |
| | | CGCtcgGCcccctga | | | |
| | | GCcGCtGCcccGCc | | | |
| | | cctGCcctGCtGCgtg | | | |
| | | ggagagGCacaggGC | | | |
| | | gaga (SEQ ID NO: | | | |
| | | 27) | | | |

Venous blood (18 ml) was collected into K₃EDTA Vacuette tubes (Greiner Bio-One, Frickenhausen, Germany) from participants. Blood tubes were kept at 4° C. prior to plasma processing (not 44 h from blood collection). Plasma was prepared by centrifugation at 1,500 g for 10 min at 4° C. (deceleration at lowest setting), followed by retrieval of the plasma fraction and a repeat centrifugation. The resulting plasma was stored at −80° C. Frozen plasma samples were shipped on dry ice to Clinical Genomics Technologies (Sydney, Australia) and stored at −80° C. until testing.

### Example 4: Analysis with an Exosome Sample

Serum was collected from subjects having stage 4 oesophogeal cancer (stage IV OEC) and from control subjects not having any cancer. Exosomes were isolated from the serum samples. The isolated exosome samples were analyzed according to the method described in Example 3 to determine the methylation levels of BCAT1 and IKZF1. High positivity rates in exosomal samples for the methylated biomarkers were observed in the cancer patients, but not in the control patients (Table 7).

TABLE 7
Exosomal Positivity for Methylated BCAT1 and IKZF1

| | Positivity rate, |
|---|---|
| | Number of Positive/Total |
| Exosomal Source | (%) |
| Healthy Control (n = 3) | 0/3 (0%) |
| Oesophogeal Subjects (n = 3) | 2/3 (67%) |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

It is understood that the examples and embodiments described herein are for illustrative purposes only. Various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

All articles, patents, patent applications, and other publications which have been cited in this disclosure are hereby incorporated herein by reference in each of their entireties.

### BIBLIOGRAPHY

Abrams and Stanton, Methods Enzymol., 212:71-74, 1992
Adorjan et al. Nucl. Acids Res., 30: e21, 2002
Alon et al., Proc. Natl. Acad. Sci. USA: 96:6745-6750, June 1999
Ammerpohl et al. Biochim Biophys Acta. 1790:847-62, 2009
Beaucage, et al. Tetrahedron Letters 22:1859-1862, 1981
Bianco et al., Hum. Mutat., 14:289-293, 1999
Bonner and Laskey, Eur. J. Biochem. 46:83, 1974
Bresslauer et al., Proc. Natl. Acad. Sci. USA, 83: 3746-3750, 1986
Caruthers, M. H., et al., Methods in Enzymology, Vol. 154, pp. 287-314 (1988)
Chen and Kwok, Nucleic Acids Res. 25:347-353, 1997
Clark et al. Nat Protoc. 1:2353-64, 2006
Clark et al., Nucl. Acids Res. 22:2990-2997, 1994
Cottrell et al., Nucl. Acids Res. 32: e10, 2003
DeGraves, et al., Biotechniques 34(1):106-10, 112-5 (2003)
Deiman B, et al., Mol. Biotechnol. 20(2):163-79 (2002)
Deng et al. Chin. J. Cancer Res., 12:171-191, 2000
Dieffenbach (ed) and Dveksler (ed) (In: PCR Primer: A Laboratory Manual, Cold Spring Harbour Laboratories, NY, 1995
Eads et al., Nucl. Acids Res. 28: E32, 2000
Egholm et al., Am. Chem. Soc., 114:1895, 1992
Egholm et al., Nature, 365:566, 1993
Fodor et al., Science 767-773, 1991
Frommer et al., Proc. Natl. Acad. Sci. USA 89:1827-1831 (1992)
Gibson et al., Genome Research 6:995-1001 (1996)
Golub et al., Science, 286:531-537, 1999
Gonzalgo & Jones, Nucleic Acids Res. 25:2529-2531 (1997)
Gonzalgo et al., Cancer Res. 57:594-599, 1997
Gregory and Feil, Nucleic Acids Res., 27, e32i-e32iv, 1999
Havelange et al., Blood 2011, 118:2827
Herman et al. Proc. Natl. Acad. Sci. USA 93:9821-9826 (1996)
Holland et al., Proc. Natl. Acad. Sci. USA, 88:7276-7280, 1991
Javierre et al., Mol. Cancer Res. 9(8):1139-51, 2011
Kawai et al., Mol. Cell. Biol. 14:7421-7427, 1994
Kibriya et al., BMC 2011, 4:50
Kristensen and Hansen Clin Chem. 55:1471-83, 2009
Kuppuswamy et al., Proc. Natl. Acad. Sci. USA 88:1143-1147, 1991
Landegren et al., Genome Res., 8(8): 769-776, 1998
Lee et al., Nucleic Acid Res. 21:3761-3766, 1993
Markowitz and Bertagnolli, 2009, N. Engl. J. Med. 361(25): 2449-60
Marmur and Doty, J. Mol. Biol. 5:109, 1962
Martinez et al., Am. J. Surg Pathol. 2012, 36:296
McPherson et al., PCR: A Practical Approach. (series eds, D. Rickwood and B. D. Hames), IRL Press Limited, Oxford. pp 1-253, 1991
Messing, Methods Enzymol, 101, 20-78, 1983
Mhlanga and Malmberg, Methods 25:463-471, 2001
Moore et al., BBA, 1402:239-249, 1988
Narang, et al. Meth. Enzymol 68: 90, 1979
Nevrivy et al. JBC 2000, 275(22):16827-36
Nielsen et al. J. Chem. Soc. Perkin Trans., 1:3423, 1997
Olek, et al. Nat. Genet. 17(3): 275-6 (1997)
Orum et al., Clin. Chem. 45:1898-1905, 1999
Orum et al., Nucl. Acids Res., 21:5332, 1993
Oster et al., Int. J. Cancer 2011, 129:2855
Pathak et al. PLoS One 2011, 6:e22628
Rand et al. Epigenetics 1:94-100, 2006
Rand et al. Nucl. Acids Res. 33:e127, 2005
Rein, et al. Nucleic Acids Res. 26 (10): 2255-64 (1998)
Sadri & Hornsby, Nucl. Acids Res. 24:5058-5059 (1996)
Sambrook et al., Molecular Cloning, A Laboratory Manual (2nd Ed., CSHP, New York 1989)
Santa Lucia, Proc. Natl. Acad. Sci. USA, 95: 1460-1465, 1995
Shames et al. Cancer Lett. 251:187-98, 2007
Simeonov and Nikiforov, Nucleic Acids Research, 30(17):1-5, 2002
Singer-Sam et al., Nucl. Acids Res. 18:687, 1990
Singer-Sam et al., PCR Methods Appl. 1: 160-163, 1992
Singh and Wengel, Chem. Commun. 1247, 1998
Slattery et al., Carcinogenesis 2011, 32:160
Southern et al., Genomics, 13:1008-1017, 1992
Szabo and Mann, Genes Dev. 9: 3097-3108, 1995
Toyota et al., Cancer Res. 59:2307-12 (1999)
Uhlmann et al., Electrophoresis, 23:4072-4079, 2002
Wedemeyer et al., Clinical Chemistry 48:9 1398-1405, 2002
Weissleder et al., Nature Medicine 6:351-355, 2000
Weitzel J N (December 1999), Cancer 86 (11 Suppl): 2483-92
Worm et al., Clin. Chem., 47:1183-1189, 2001
Xiong & Laird, Nucleic Acids Res. 25:2532-2534 (1997)
Yamashita et al. (Cancer Sci. 2010, 101:1708)
Zyskind et al., Recombinant DNA Laboratory Manual, (Acad. Press, 1988)

1. A method of screening for the onset or predisposition to the onset of or monitoring an esophageal or gastric neoplasm in an individual, said method comprising assessing the methylation status of a DNA region selected from:
(i) the region, including 2 kb upstream of the transcription start site, defined by Hg19 coordinates:
(1) chr12:24962958 . . . 25102393; and/or
(2) chr7:50344378 . . . 50472798
or
(ii) the gene region, including 2 kb upstream of any two or more of:
(1) BCAT1 and/or (2) IKZF1
in a biological sample from said individual wherein a higher level of methylation of at least one of the DNA regions of group (i) and/or (ii) relative to control levels is indicative of an esophageal or gastric neoplasm or a predisposition to the onset of a esophageal or gastric neoplasm.

2. (canceled)

3. The method according to claim 1, wherein said method is directed to screening either BCAT1 or IKZF1 in said biological sample.

4. The method according to claim 1, said method is directed to screening both BCAT1 and IKZF1 in said biological sample.

5. The method according to claim 4, wherein only one of said BCAT1 and IKZF1 exhibits modulated methylation.

6. The method according to claim 4, wherein both of said BCAT1 and IKZF1 exhibit modulated methylation or.

7. The method according to claim 1, wherein the neoplasm is malignant.

8. The method according to claim 7, wherein said malignant neoplasm is an adenocarcinoma.

9. The method according to claim 1, wherein said neoplasm is not malignant.

10. The method according to claim 9, wherein said non-malignant neoplasm is an adenoma.

11. The method according to claim 1, wherein said control level is a non-neoplastic level.

12. The method according to claim 1, wherein said control level is the level of a previously screened biological sample from said individual.

13. The method according to claim 12, wherein a decrease in the level of methylation relative to said control level is indicative of the clearing of the neoplasm.

14. The method according to claim 1, wherein said neoplasm is a gastric neoplasm.

15. The method according to claim 1, wherein said neoplasm is an esophageal neoplasm.

16. The method according to claim 1, wherein said biological sample is a surgical resection, tissue biopsy, saliva, urine or blood sample.

17. The method according to claim 16, wherein said blood sample is whole blood, serum, plasma, exosomes, or buffy coat.

18. The method according to claim 17, wherein the DNA methylation screening is directed to cell free DNA.

19. The method according to claim 18, wherein said cell free DNA is circulating tumor DNA.

20. The method according to claim 1, wherein said methylation is assessed in one or more chromosomal subregions selected from:
(1) BCAT1 subregions chr12:25101992-25102093 (SEQ ID NO: 9 or corresponding minus strand) and chr12:25101909-25101995 (SEQ ID NO: 16 or corresponding minus strand);
(2) IKZF1 subregions: chr7:50343867-50343961 (SEQ ID NO: 2 or corresponding minus strand) and chr7:50343804-5033895 (SEQ ID NO: 24 or corresponding minus strand).

21. The method according to claim 20, said method comprising assessing the methylation of one or more cytosine residues selected from:
(IKZF1)

| | | |
|---|---|---|
| chr7: 50343869 | chr7: 50343872 | chr7: 50343883 |
| chr7: 50343889 | chr7: 50343890 | chr7: 50343897 |
| chr7: 50343907 | chr7: 50343909 | chr7: 50343914 |
| chr7: 50343934 | chr7: 50343939 | chr7: 50343950 |
| chr7: 50343959 | chr7: 50343805 | chr7: 50343822 |
| chr7: 50343824 | chr7: 50343826 | chr7: 50343829 |
| chr7: 50343831 | chr7: 50343833 | chr7: 50343838 |
| chr7: 50343847 | chr7: 50343850 | chr7: 50343858 |
| chr7: 50343864 | chr7: 50343869 | chr7: 50343872 |
| chr7: 50343890 | | |

or a corresponding cytosine at position n+1 on the opposite DNA strand.

22. (canceled)

23. The method according to claim 1, wherein said mammal is a human.
